(19) United States
(12) Patent Application Publication
(10) Pub. No.: US 2013/0281523 A1
(43) Pub. Date: Oct. 24, 2013

(54) LOW DOSE CANNABINOID MEDICAMENTS

(71) Applicants: Peter Letendre, Lafayette, CO (US); David Carley, Evanston, IL (US)

(72) Inventors: Peter Letendre, Lafayette, CO (US); David Carley, Evanston, IL (US)

(21) Appl. No.: 13/261,662

(22) PCT Filed: Nov. 18, 2011
(86) PCT No.: PCT/US11/61490

§ 371 Date: May 21, 2013

Related U.S. Application Data
(60) Provisional application No. 61/415,331, filed on Nov. 18, 2010.

(51) Int. Cl. A61K 31/352; A61K 9/00; A61K 45/06

(52) U.S. Cl. CPC A61K 31/352; A61K 45/06; A61K 9/0053
USPC 514/454; 549/390

(57) ABSTRACT

The present invention provides methods for treating cannabinoid-sensitive disorders with a lose-dose oral cannabinoid which results in delivery of a therapeutic level during an extended clinically-relevant therapeutic window. These methods provide therapeutic dosing while maintaining safe, side effect sparing, levels of a cannabinoid. The present invention also provides methods of determining optimal dosing in treated patients.

### TECHNICAL FIELD

The present invention relates to cannabinoid compositions and methods of treating cannabinoid-sensitive disorders (e.g. apnea) with cannabinoids.

### BACKGROUND

Over the past several years, much effort has been devoted to the study of a discrete group of breathing disorders that occur primarily during sleep with consequences that may persist throughout the waking hours, most commonly in the form of sleepiness and/or cognitive/motor impairment, thereby manifesting itself into substantial economic loss (e.g., thousands of lost man-hours) or employment safety factors (e.g., employee non-attentiveness during operation of heavy-machinery). Sleep-related breathing disorders are characterized by repetitive reduction in breathing (hypopnea), cessation of breathing (apnea), or a continuous or sustained reduction in ventilation, (hypoventilation).

In general, sleep apnea is defined as an intermittent cessation of airflow at the nose and mouth during sleep. By convention, apneas of at least 10 seconds in duration have been considered important, but in most individuals the apneas are 20-30 seconds in duration and may be as long as 2-3 minutes. While there is some uncertainty as to the minimum number of apneas that should be considered clinically important, by the time most individuals come to attention of the medical community they have at least 10 to 15 events per hour of sleep.

Sleep apneas have been classified into three types: central, obstructive, and mixed. In central sleep apnea the neural drive to all respiratory muscles is transiently abolished. In obstructive sleep apneas, airflow ceases despite continuing respiratory drive because of occlusion of the oropharyngeal airway. Mixed apneas, which comprise a central apnea followed by an obstructive component, are a variant of obstructive sleep apnea. The most common type of apnea is obstructive sleep apnea. Although airflow persists during hypopneas, like apneas they are associated with reduced oxygen levels in the arterial blood and/or arousals from sleep. Apneas and hypopneas are viewed as carrying equal clinical significance. Because airflow persists, hypopneas are not classified as either central or obstructive.

Obstructive sleep apnea syndrome (OSAS) has been identified in as many as 24% of working adult men and 9% of similar women, with peak prevalence in the sixth decade of life. Habitual heavy snoring, which is an almost invariant feature of OSAS, has been described in up to 24% of middle aged men, and 14% of similarly aged women, with even greater prevalence in older subjects.

Obstructive sleep apnea syndrome's definitive event is the occlusion of the upper airway, frequently at the level of the oropharynx. The resultant apnea generally leads to a progressive-type asphyxia until the individual is briefly aroused from the sleeping state, thereby restoring airway patency and thus restoring airflow.

An important factor that leads to the collapse of the upper airway in OSAS is the generation of a critical subatmospheric pressure during the act of inspiration that exceeds the ability of the airway dilator and abductor muscles to maintain airway stability. Sleep plays a crucial role by reducing the activity of the muscles of the upper airways including the dilator and abductor muscles.

In most individuals with OSAS, the patency of the airway is also compromised structurally and is therefore predisposed to occlusion. In a minority of individuals the structural compromise is usually due to obvious anatomic abnormalities, i.e, adenotonsillar hypertrophy, retrognathia, or macroglossia. However, in the majority of individuals predisposed to OSAS, the structural abnormality is simply a subtle reduction in airway size, i.e., “pharyngeal crowding.” Obesity also frequently contributes to the reduction in size seen in the upper airways. The act of snoring, which is actually a high-frequency vibration of the palatal and pharyngeal soft tissues, usually aggravates the narrowing via the production of edema in the soft tissues.

The recurrent episodes of nocturnal asphyxia and of arousal from sleep that characterize OSAS lead to a series of secondary physiologic events, which in turn give rise to the clinical complications of the syndrome. The most common manifestations are neuropsychiatric and behavioral disturbances that are thought to arise from the fragmentation of sleep and loss of slow-wave sleep induced by the recurrent arousal responses. Nocturnal cerebral hypoxia also may play an important role. The most pervasive manifestation is excessive daytime sleepiness, although insomnia is common in the elderly with OSAS. OSAS is now recognized as a leading cause of daytime sleepiness and has been implicated as an important risk factor for such problems as motor vehicle accidents. Other related symptoms include intellectual impairment, memory loss, personality disturbances, and impotence.

Other major manifestations are cardiorespiratory in nature and are thought to arise from the recurrent episodes of nocturnal asphyxia. Most individuals demonstrate a cyclical slowing of the heart during the apneas to 30 to 50 beats per minute, followed by tachycardia of 90 to 120 beats per minute during the ventilatory phase. A small number of individuals develop severe bradycardia with asystoles of 8 to 12 seconds in duration dr dangerous tachyarrhythmias, including unsustained ventricular tachycardia. OSAS also aggravates left ventricular failure in patients with underlying heart disease. This complication is most likely due to the combined effects of increased left ventricular afterload during each obstructive event, secondary to increased negative intrathoracic pressure, recurrent nocturnal hypoxemia, and chronically elevated sympathoadrenal activity.

Central sleep apnea is less prevalent as a syndrome than OSAS, but can be identified in a wide spectrum of patients with medical, neurological, and/or neuromuscular disorders associated with diurnal alveolar hypoventilation or periodic breathing. The definitive event in central sleep apnea is transient abolition of central drive to the ventilatory muscles. The resulting apnea leads to a primary sequence of events similar to those of OSAS. Several underlying mechanisms can result in cessation of respiratory drive during sleep. First are defects in the metabolic respiratory control system and respiratory neuromuscular apparatus. Other central sleep apnea disorders arise from transient instabilities in an otherwise intact respiratory control system.

Many healthy individuals demonstrate a small number of central apneas during sleep, particularly at sleep onset and in REM sleep. These apneas are not associated with any physiological or clinical disturbance. In individuals with clinically significant central sleep apnea, the primary sequence of events that characterize the disorder leads to prominent physiological and clinical consequences. In those individuals with central sleep apnea alveolar hypoventilation syndrome, daytime hypercapnia and hypoxemia are usually evident and the clinical picture is dominated by a history of recurrent respiratory failure, polycythemia, pulmonary hypertension, and right-sided heart failure. Complaints of sleeping poorly, morning headache, and daytime fatigue and sleepiness are also prominent. In contrast, in individuals whose central sleep apnea results from instability in respiratory drive, the clinical picture is dominated by features related to sleep disturbance, including recurrent nocturnal awakenings, morning fatigue, and daytime sleepiness.

Currently, the most common and most effective treatments, for adults with sleep apnea and other sleep-related breathing disorders, are mechanical forms of therapy that deliver positive airway pressure (PAP). Under PAP treatment, an individual wears a tight-fitting plastic mask over the nose when sleeping. The mask is attached to a compressor, which forces air into the nose creating a positive pressure within the patient's airways. The principle of the method is that pressurizing the airways provides a mechanical “splinting” action, which prevents airway collapse and therefore, obstructive sleep apnea. Although an effective therapeutic response is observed in most patients who undergo PAP treatment, many patients cannot tolerate the apparatus or pressure and refuse treatment. Moreover, covert monitoring studies clearly demonstrate that long-term compliance with PAP treatment is very poor. A variety of upper airway and craniofacial surgical procedures have been attempted for treatment of OSAS. Adenotonsillectomy appears to be an effective cure for OSAS in many children, but upper airway surgery is rarely curative in adult patients with OSAS. Surgical “success” is generally taken to be a 50% reduction in apnea incidence and there are no useful screening methods to identify the individuals that would benefit from the surgery versus those who would not derive a benefit.

Recently, the present inventors demonstrated that intraperitoneal injection of THC at 10 mg/kg in a rat model of sleep apnea reduced breathing cessation during non-REM (“NREM”) sleep as described in US 2004/0127572. What is needed in the art is a convenient oral medicament in an amount that reduces apnea.

Also needed is an oral dosage that is effective yet minimizes undesirable effects (e.g. of inducing psychotropic responses). Also needed is a medicament that provides therapeutic efficacy for a period of time roughly equivalent to a typical human sleep period (e.g. about 6 to 8 hours) and that doesn't require repeated dosage through that period. Also needed is an oral medicament that allows the subject to wake from sleep without residual side effects that negatively impact wakefulness and alertness without other known affects such as a overly-stimulated appetite.

### SUMMARY OF THE INVENTION

The present invention provides methods of administering low dose cannabinoid compositions (medicaments) to a subject with a cannabinoid-sensitive disorder, resulting in delivery of a therapeutic level of one or more cannabinoids during a clinically relevant therapeutic window. The present low dose compositions provide therapeutic dosing at levels to generally'avoid levels typically associated with certain side effects.

Examples of cannabinoid-sensitive disorders are sleep apnea, anxiety, stress, headache, nausea, glaucoma, pain, arthritis, irritable bowel syndrome, ulcerative colitis, Crohn's disease, anorexia or cachexia syndrome, bladder dysfunction, spasticity due to multiple sclerosis, Huntington's disease, and Alzheimer's disease.

The present medicaments provide dosing, for example, oral dosing of about 0.05 to about 25 mg of a cannabinoid. Optionally, the subject sleeps during the therapeutic window.

In each embodiment of the present invention, the cannabinoid is optionally dronabinol (e.g. oral dronabinol).

Optionally, the oral medicaments of the present invention provide a therapeutic response without causing, or while causing only mild, side effects associated with cannabinoids. Optionally, the oral medicaments of the present invention, when administered to a subject immediately before a sleep cycle, provide a therapeutic response without causing (or without substantially causing) side effects once the subject has awoken (e.g. post treatment window).

In one embodiment, a subject with a cannabinoid-sensitive disorder is treated with a cannabinoid dose for a prolonged treatment period and then treated with the cannabinoid dose for a subsequent treatment period, wherein the therapeutic efficacy during the subsequent treatment period is greater than the therapeutic efficacy during the prolonged treatment period. Optionally, the prolonged treatment period is at least 30 days. Optionally, the cannabinoid dose is about 0.05 to about 5 mg (e.g. 0.05 mg to about 2.0 mg).

In one embodiment, a subject with a cannabinoid-sensitive disorder is treated with a first cannabinoid dose for a first treatment period followed by a second treatment period comprising a second cannabinoid dose, reduced as compared to the first cannabinoid dose, wherein therapeutic efficacy in the second treatment period is not reduced compared to the therapeutic efficacy during the first treatment period. Optionally, the first and second cannabinoid doses are about 0.05 to about 5 mg. In one embodiment, the method provides treatment over a prolonged treatment period, e.g. more than about one week or more than about 1 month or more than about 1 year.

The present invention also provides methods of determining optimal dosing in treated patients.

### BRIEF DESCRIPTION OF THE DRAWINGS

FIG. 1 depicts the % of subjects with a 75% reduction in AHI versus duration of reduction: the effect of THC dose.

FIG. 2 depicts dose and time dependent effects of THC on apnea suppression.

FIG. 3 depicts AHI in sleep apnea patients during a target treatment window.

FIG. 4 depicts the relationship of Cmax (ng/ml) and cannabinoid amount (mg) for an immediate release compartment (Marinol formulation).

FIG. 5 depicts plasma profiles depicted in US 2009/0181080 of a THC formulation.

FIG. 6 depicts the efficacy of present medicaments with weeks of treatment.

### DETAILED DESCRIPTION OF THE INVENTION

As used here, the following definitions and abbreviations apply.

“AHI” means apnea-hypopnea index, which is calculated by dividing the number of apnea and hypopnea events by the number of hours of sleep. The AHI index generally quantifies the overall severity of sleep apnea including sleep disruptions and desaturations. Typically, an AHI of 5-15 is considered mild, 15-30 is moderate, and above 30 is severe.

“AUC” (area-under-the-curve) is the overall amount of THC (or metabolite thereof) in the bloodstream or plasma after a dose. AUC can be calculated by collecting multiple blood samples over a period of time, graphing the drug concentrations, and calculating the drug as the area under this drug concentration curve. AUC can be expressed in units of amount of THC×time/volume (e.g. ng×hr/ml).

“Cmax” means the maximum plasma concentration of THC (or a metabolite thereof) during an interval of time.

“Cannabinoid-sensitive disorder” means a disorder that, when a cannabinoid or a cannabinoid receptor modulator is administered, modulates a pathophysiologic pathway that ameliorates the disorder or clinically relevant symptoms thereof. Relevant pathophysiologic pathways can be desirably modulated by present medicaments. For example, administration may modulate the pathways of acid (e.g. GABA, glutamate), monoamine (e.g. histamine, dopamine, serotonin, noradrenaline) purine (e.g. adenosine, ADP, ATP), peptide (e.g. somatostatin, neuropeptide Y, neurokinin, cholecystokinin), vanilloid, prostanoid, opiod and/or other neurotransmitters. Accordingly, cannabinoid-sensitive disorders include disorders mediated by or sensitive to neurotransmitter action.

“Cmin” (or trough) is the lowest concentration of THC (or a metabolite thereof) in the plasma (following the Cmax) within a defined treatment window.

“Examplary” (or “e.g.” or “by example”) means a non-limiting example.

“Immediate release dosage compartment” (or “IR”) means a dosage compartment that does not contain a release modifier in a release modifying amount.

“Marinol” means a gel capsule medicament of dronabinol as it generally is formulated and available under the trademark MARINOL®. Where reference is made to Marinol at a concentration that is not commercially available, it is meant to refer to a medicament formulated similarly to other strengths of MARINOL®, i.e. containing dronabinol, gelatin, glycerin, and sesame oil.

“Substantially similar”, as it relates to a referenced quantifiable parameter (e.g. THC plasma level, Cmax, Tmax, or therapeutic response) means that the subject parameter is from about 50% to about 200% of the referenced parameter, or from about 75% to about 150%, or about 80% to about 120%.

“Therapeutic window” means a period of time during which a therapeutically effective level of drug is maintained.

“Treatment window” means the period of time beginning at the time of administration (i.e. T₀) of the drug composition and ending at a defined time. The treatment window can be further divided into sub-periods, such as “early treatment window” (e.g. T₁ₕᵣ-T₄ₕᵣ or T₁ₕᵣ-T₅ₕᵣ) or “late treatment window” (e.g. T₄ₕᵣ-T₈ₕ, or T₃ₕᵣ-T₈). The units of subscript of “T”, where not stated, are “hours” (for example, T₁=T₁ₕᵣ).

“Therapeutic efficiency” means the ratio of therapeutic response to side effects (i.e. any treatment-related effects that are not a therapeutic response).

“Therapeutic response” means any response that can be considered to represent a reduction in the signs or symptoms of a medical condition. For apnea, a therapeutic response is, for example, a reduction in apnea-hypopnea index, snoring, oxygen desaturation of the arterial blood, or sleep disruption.

“Tmax” means the time between the administration of the medicament and the time that a maximum plasma level (Cmax) of the referenced cannabinoid (or metabolite) is achieved.

Present Medicaments for Cannabinoid Sensitive Disorders

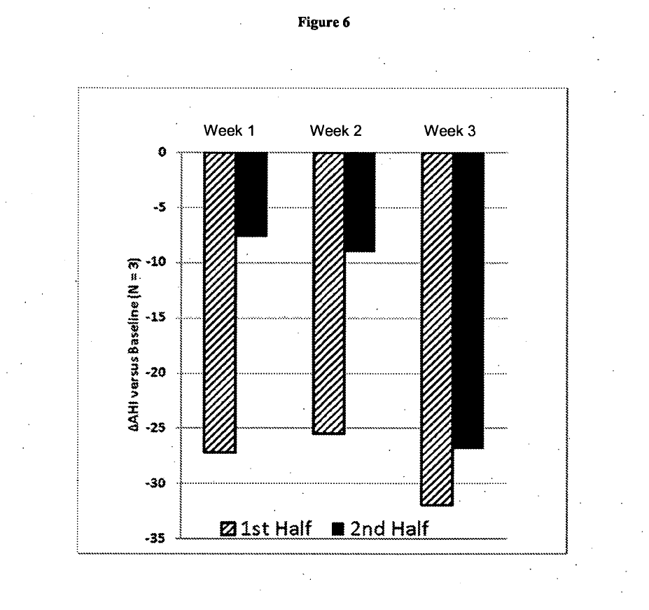

The present medicaments are surprisingly effective for treating certain cannabinoid sensitive disorders. Technical features include providing, when administered so certain subjects: (1) a therapeutic window which begins within about 30 minutes or about 1 hour or about 2 hours of administrations (e.g. as shown by Example 3); (2) a therapeutic window that is about 1 to about 8 hours or to about 12 hours long (e.g. as shown in FIG. 6); and (3) plasma levels that do not elevate into a level where reduced therapeutic efficacy and/or deleterious side effects are produced. (e.g. as shown in Example 3, it has been surprisingly discovered that certain patients with cannabinoid-sensitive disorders exhibit a non-monotonic dose-response of the inverted U type).

Cannabinoids

The compositions of the present invention provide one or more cannabinoids in a medicament that can deliver to a subject a desired target PK profile, where the PK profile achieves a therapeutic level of a cannabinoid during a therapeutic window. Cannabinoids of the present invention are any member of a group of substances that are structurally related to tetrahydrocannabinol and that bind to a cannabinoid receptor such as CB1 or CB2 or both (‘THC’). The cannabinoid can be a naturally occurring compound (e.g. present in Cannabis), a compound metabolized by a plant or animal, or a synthetic derivative.

The cannabinoid may be included in its free form, or in the form of a salt; an acid addition salt of an ester; an amide; an enantiomer; an isomer; a tautomer; a prodrug; a derivative of an active agent of the present invention; different isomeric forms (for example, enantiomers and diastereoisomers), both in pure form and in admixture, including racemic mixtures; enol forms.

The cannabinoids of the present invention are further meant to encompass natural cannabinoids, natural cannabinoids that have been purified or modified, and synthetically derived cannabinoids, for example, United States Patent Application Publication 2005/0266108, hereby incorporated by reference in its entirety, describes a method of purifying cannabinoids obtained from plant material.

The cannabinoids of the present invention can be any of 9-tetrahydrocannabinol, 8-tetrahydrocannabinol, (+)-1,1-dimethylheptyl analog of 7-hydroxy-delta-6-tetrahydrocannabinol, 3-(5′-cyano-1′,1′-dimethylpentyl)-1-(4-N-morpholinobutyryloxy) delta 8-tetrahydrocannabinol hydrochloride], dexanabinol, nabilone, levonantradol, or N-(2-hydroxyethyl)hexadecanoamide. The cannabinoids of the present invention can be any of the non-psychotropic cannabinoid 3-dimethylnepty 11 carboxylic acid homologine 8, delta-8-tetrahydrocannabinol. (J. Med. Chem. 35, 3135, 1992).

The cannabinoids of the present invention can further be any of the active metabolites, derivatives, or analogs as taught in the National Institute on Drug Abuse Research Monograph Series 79, “Structure-Activity Relationships of the Cannabinoids.

In each of the embodiments of the present invention, the cannabinoid can be Delta-9-tetrahydrocannabinol, also known as dronabinol. Dronabinol is naturally-occurring and has been extracted from Cannabis sativa L. (marijuana). It has also been produced chemically as described in U.S. Pat. No. 3,668,224. Dronabinol is a light-yellow resinous oil that is sticky at room temperature, but hardens upon refrigeration. It turns to a flowable liquid when heated at higher temperatures. Dronabinol is insoluble in water and typically formulated in sesame oil. It has a pKa of 10.6 and an octanol-water partition coefficient: 6,000:1 at pH 7. Dronabinol is available in natural (extracted from plant) and synthetic forms. On the other hand, synthetic dronabinol may be utilized and may be synthesized using the starting materials: Olivetol and p-2,8-menthadien-2-ol (PMD).

The term “dronabinol” is further meant to encompass naturally occurring dronabinol, synthetically derived dronabinol, and synthetically modified dronabinol starting with a molecule obtained from a natural source for example, United States Patent Application Publication 2005/0171361, hereby incorporated by reference in its entirety, describes a method of extracting delta-9-THC acid from the plant material by chromatography and then synthetically converting it to dronabinol.

Structural features of the present cannabinoids, and structure-function relationships are well-known in the art and taught, for example, by Rao Rapaka and Alexandros Makriyannis in “Structure-Activity Relationships of the Cannabinoids”, NIDA Research Monograph 79 (1987).

Medicaments

With the teachings provided herein, one skilled in the art can now administer a medicament that, when provided orally to a subject, produces a therapeutic response over a desired therapeutic window (e.g. extending over both an early treatment window and a late treatment window). In some embodiments, the formulations minimize the total amount of drug administered, thus significantly decreasing side effects and increasing the therapeutic efficiency.

The component(s) of the medicaments may be in any form, e.g. liquid, solid, and semi-solid components.

The appropriate selection and amount of excipients is often influenced by the selection and amount (or fraction of the total dose) of cannabinoid(s) associated (e.g. compounded) with the excipients in a component (e.g. microparticle) of the dosage (and vice versa).

The medicaments of the present invention can be made with different polymorphic forms (e.g. salts, crystalline forms, hydrates, esters, and solvates), each with physicochemical properties affecting drug delivery (e.g. absorption). Selection of the release modifiers is done with consideration of the THC form. A number of such forms are well known in the art.

For example, in one embodiment of the invention, the cannabinoid form used in the formulation is a cannabinoid ester or salts thereof (e.g. a polar ester such as an ester of a terminal carboxylic acid). Esterified forms of THC are described, for example, in U.S. Pat. No. 4,933,368, U.S. Pat. No. 5,389,375 and U.S. Pat. No. 6,008,383. Other useful polar esters are the hemi-ester of malonic acid and the alaninate ester of alanine. It has been reported, e.g., in U.S. Pat. No. 5,508,051 and U.S. Pat. No. 5,389,375, that salts of the terminal carboxylic acid group of the ester, for example, the N-methyl glutamine salt as well as the sodium and potassium salts are also useful.

In another example, the cannabinoid form used in the formulation is in crystalline form. Optionally, the cannabinoid form is crystalline trans-(+/−)-THC. Examples of such crystalline forms are described, for example, in US 2007/0072939.

In each embodiment taught herein, the medicament is optionally an IR medicament.

Liquid Dosage Compartments

In one embodiment, a dosage compartment of the present composition is a liquid or predominantly liquid (a liquid) dosage compartment. Any formulation useful for oily or lipophilic compounds may be used. For example, the component may be in the form of an aqueous or non-aqueous liquid, an oil or other lipophilic medium, an emulsion, a syrup, and the like. Optionally, a liquid compartment is encapsulated (e.g. a hard gel or soft gel).

Semi-Solid Dosage Compartments

In one embodiment, a dosage compartment of the present composition is a semi-solid dosage compartment. Any formulation useful for oily or lipophilic compounds may be used. For example, the compartment may be in the form of self-emulsifying drug delivery system (SEDDS), or a lipophilic medium compartment. Optionally, the semi-solid compartment is encapsulated (e.g. a hard gel or soft gel).

Solid Dosage Compartments

In one embodiment, a dosage compartment of the present invention is a solid dosage compartment. Any formulation useful for oily or lipophilic compounds may be used. For example, the dosage compartment may be a solid lipid dosage compartment, a solid dosage compartment produced from an aqueous mixture or emulsion, a solid dosage compartment produced by extrusion (e.g. hot melt extrusion), or a solid emulsion that is, for example, dried. Other solid dosage compartments include osmotic particles. Optionally, a solid dosage compartment (e.g. powdered, spray dried, or freeze dried forms) is formed into a tablet, pill, microsphere, and the like.

Optional Excipients

Component and/or composition properties, for example, bulk stability, dissolution and other release properties of composition components (e.g. solid, liquid, or semi-solid dosage compartments) may be manipulated by choosing an appropriate excipient, amount thereof, or formulation method using such.

Combinations

The present medicaments can optionally be combined with one or more additional therapeutic agents.

The present medicaments can optionally be combined with therapeutic agents useful in the treatment of a cannabinoid-sensitive disorder. For example, the one or more additional therapeutic agents are optionally therapeutic agents useful in the treatment of cannabinoid-sensitive disorder selected from: apnea, seizures, a neurological disorder, a pain disorder, an appetite or wasting disorder, nausea, vomiting, a sleep disorder, a breathing disorder, or a sleep-related breathing disorder.

Optionally, the present medicaments are combined with one or more anti-apnea therapeutic agents, for example, any of: serotonin reuptake inhibitors, serotonin receptor antagonists, serotonin receptor (e.g. subtype 1) agonists, serotonin agonists, noradrenalin reuptake inhibitors, combined serotonin/noradrenalin reuptake inhibitors, glutamate receptor antagonists, glutamate antagonists, inhibitors of glutamate release, glycine antagonists, GABA receptor agonists, calcitonin gene-related peptide (CGRP) receptor antagonists or release inhibitors, adenosine, adenosine analogs and nucleoside (e.g. adenosine) uptake blockers or reuptake inhibitors, opioid antagonists, vanilloid receptor ligands, pilocarpine compounds, sodium proton pump inhibitors, ubidecarenones, antihistimines, prostaglandins, prostanoid receptor antagonists, inhibitors of prostanoid synthesis, modulators of CRTH2, COX-2 and/or FAAH, antitussive agents, compounds that stimulate the central nervous system, agents that prolong the action of endocannabimimetics, inhibitors of endocannabinoid membrane transport, inhibitors of cannabinoid metabolism, and cannabinoid degradative enzyme antagonists.

Optionally, the present medicaments are combined with one or more anti-apnea therapeutic agents or combinations of apnea therapeutic agents disclosed in any of: U.S. Pat. No. 6,331,536, U.S. Pat. No. 6,555,564, U.S. Pat. No. 6,727,242, U.S. Pat. No. 7,160,898, U.S. Pat. No. 6,974,814, U.S. Pat. No. 7,705,039, US20060241164, US20070123517, US20080200367, US20080261922, US20090005357, US20090221658, US20100137251, EP1066036, EP1372638, EP1572202, EP1868602, JP2010059195, WO/1999/043319, WO/2000/051590, WO/2002/080903, WO/2004/041272, WO/2006/113448, WO/2006/133197, WO/2007/047372, WO/2007/047575, WO/2007/047576, WO/2007/047577, which disclose useful examples of the anti-apnea therapeutic agents and/or other anti-apnea therapeutic agents.

Optionally, the present medicaments are combined with one or more anti-convulsants, for example, anti-convulsants of any of the following types: aldehydes, aromatic allylic alcohols, barbiturates, benzodiazepines, bromides, carbamates, carboxamides, fatty acids, fructose derivatives, gaba analogs, hydantoins, oxazolidinediones, propionates, pyrimidinediones, pyrrolidines, succinimides, sulfonamides, triazines, ureas, and valproylamides (amide derivatives of valproate).

Optionally, the present medicaments are combined with one or more analgesics, for example, any of: NSAIDs (e.g. ibuprofen), paracetamol, COX-1 inhibitors, COX-2 inhibitors, COX-3 inhibitors, opioids (e.g. hydrocodone or oxycodone), morphinomimetics, flupirtine, tricyclic antidepressants (e.g. amitriptyline), tetracyclic antidepressants, anticonvulsants (e.g. carbamazepine, gabapentin, or pregabalin), anticholinergics, antispasmodics, K+ channel openers, NMDA receptor antagonists (e.g, dextromethorphan, ketamine, and amantadine), steroids, anti-inflammatories, non-narcotic analgesic (e.g. tramadol), NK1 receptor antagonists (e.g., ezlopitant and SR-14033, SSR-241585), CCK receptor antagonists (e.g., loxiglumide), NK3 receptor antagonists (e.g., talnetant, osanetant SR-142801, SSR-241585), norepinephrine-serotonin reuptake inhibitors (NSR1; e.g., milnacipran), vanilloid receptor agonists and antagonists, cannabinoid receptor agonists (e.g., arvanil), sialorphin, inhibitors of neprilysin, CCK receptor agonists (e.g., caerulein), SSRIs (e.g. fluoxetine, paroxetine, or sertraline), serotonin receptor agonists, serotonin receptor antagonists, triptans (e.g. sumatriptan), GABA analogs (e.g. GABApentin or pre-gabalin), muscle relaxants, alpha-adrenergic, PDEV inhibitors, PDEVII inhibitors, and glycine antagonists.

Optionally, the present medicaments are combined with one or more anxiolytic or anti-anxiety therapeutic agents, for example, any of: benzodiazepines, buspirone, tricyclic antidepressants, SSRIs, monoamine oxidase inhibitors, antipsychotic agents, antihistamines (e.g. Atarax or Vistaril), barbiturates (e.g. phenobarbital), and beta-blockers (e.g. propranolol), and propanediols (e.g. meprobamate).

Optionally, the present medicaments are combined with one or more anti-wasting therapeutic agents or appetite stimulants, for example, any of: tricyclic antidepressants, tetracyclic antidepressants, cyproheptadine, buclizine, megestrol, ginger, EPA (fish oil), erthythropoietin, thalidomide, ghrelin, interferon, melatonin, non-steroidal anti-inflammatories, nandrolone, antidepressants, atypical antipsychotics such as olanzapine, dexamethasone, prednisolone and methylprednisolone.

Optionally, the present medicaments are combined with one or more anti-glaucoma therapeutic agents, e.g., fish oil and omega 3 fatty acids, bilberries, vitamin E, cannabinoids, camitine, coenzyme Q10, curcurmin, Salvia miltiorrhiza, dark chocolate, erythropoietin, folic acid, Ginkgo biloba, Ginseng, L-glutathione, grape seed extract, green tea, magnesium, melatonin, methylcobalamin, N-acetyl-L cysteine, pycnogenols, resveratrol, quercetin and salt, magnesium, ginkgo, salt and fludrocortisone.

Optionally, the present medicaments are combined with one or more anti-emetics, e.g., serotonin receptor antagonist, dopamine antagonist, NK1 receptor antagonist, antihistamine, benzodiazepine, anticholinergic, or steroid such as dexamethasone.

Optionally, the present medicaments are combined with one or more additional therapeutic agents selected from: antihistamines, antimicrobial agents, fungistatic agents, germicidal agents, hormones, antipyretic agents, antidiabetic agents, bronchodilators, antidiarrheal agents, antiarrhythmic agents, coronary dilation agents, glycosides, spasmolytics, antihypertensive agents, antidepressants, antianxiety agents, antipsychotic agents, other psychotherapeutic agents, steroids, corticosteroids, analgesics, cold medications, vitamins, sedatives, hypnotics, contraceptives, nonsteroidal anti-inflammatory drugs, blood glucose lowering agents, cholesterol lowering agents, anticonvulsant agents, other antiepileptic agents, immunomodulators, anticholinergics, sympatholytics, sympathomimetics, vasodilatory agents, anticoagulants, antiarrhythmics, prostaglandins having various pharmacologic activities, diuretics, sleep aids, antihistaminic agents, antineoplastic agents, oncolytic agents, antiandrogens, antimalarial agents, and antileprosy agents.

The present medicaments can optionally be combined with one or more SSRIs, e.g., fluoxetine, paroxetine, fluvoxamine, sertraline, citalopram, norfluoxetine, r(−) fluoxetine, s(+) fluoxetine, demethylsertraline, demethylcitalopram, venlafaxine, milnacipran, sibutramine, nefazodone, R-hydroxynefazodone, (−)venlafaxine, and (+) venlafaxine.

The present medicaments can optionally be combined with one or more serotonin receptor antagonists, e.g., the free base form or a quaternized form of zatosetron, tropisetron, dolasetron, hydrodolasetron, mescaline, oxetorone, homochlorcyclizine, perlapine, ondansetron (GR38032F), ketanserin, loxapine, olanzapine, Chlorpromazine, haloperidol, r (+) ondansetron, cisapride, norcisapride, (+) cisapride, (−) cisapride, (+) norcisapride, (−) norcisapride, desmethylolanzapine, 2-hydroxymethylolanzapine, 1-(2-fluorophenyl)-3-(4-hydroxyaminoethyl)-prop-2-en-1-one-O-(2-dimethylaminoethyl)-oxime, risperidone, cyproheptadine, clozapine, methysergide, granisetron, mianserin, ritanserin, cinanserin, LY-53,857, metergoline, LY-278,584, methiothepin, p-NPPL, NAN-190, piperazine, SB-206553, SDZ-205,557, 3-tropanyl-indole-3-carboxylate, 3-tropanyl-indole-3-carboxylate methiodide, and other serotonin receptor antagonists and their quaternized forms or one of its pharmaceutically acceptable salts.

The present medicaments can optionally be combined with one or more serotonin receptor agbnists, e.g., 8-OH-DPAT, sumatriptan, L694247 (2-[5-[3-(4-methylsulphonylamino)benzyl-1,2,4-oxadiazol-5-yl]-1H-indol-3-yl]ethanamine), buspirone, alnitidan, zalospirone, ipsapirone, gepirone, zolmitriptan, risatriptan, 311C90, α-Me-5-HT, BW723C86 (1-[5(2-thienylmethoxy)-1H-3-indolyl[propan-2-amine hydrochloride), and MCPP (m-chlorophenylpiperazine).

The present medicaments can optionally be combined with one or more a 2 adrenergic receptor antagonists, e.g., phenoxybenzamine, phentolamine, tolazoline, terazosine, doxazosin, trimazosin, yohimbine, indoramin, ARC239, and prazosin.

The present medicaments can optionally be combined with one or more noradrenalin reuptake inhibitors, e.g. desipramine, nortriptyline, reboxetine, nisoxetine, atomoxetine, or LYI 39603 (tomoxetine).

The present medicaments can optionally be combined with one or more antitussive agents, e.g., RSD931, FK888, CP99994, SR48968, codeine, SR142801, SB235375, nociceptin/orphanin FQ, 15-HPETE, NADA, anandamide, lidocaine, benzonatate, mexiletine, NS 1619, furosemide, zafirlukast, HOE140, dihydrocodone, oxycodone, BW443C noscapine, dextromethorphan, SKF10047, baclofen, diphenhydramine, caramiphen, glaucine, cilomilast, RO-64-6198, NKP608, levchromkalim, BRL55834, icatibant, suplatast tosilate, epinastine, levodropropizine, and other antitussive agents {Trends in Pharm Sci 25: 569-576, 2004; Br J Pharmacol 117:853-858, 1996).

The present medicaments can optionally be combined with one or more CCK receptor antagonists, e.g. CCK A receptor antagonist, a CCK B receptor antagonists, or an antagonist exhibits activity against both CCK A and CCK B receptors. Examplary antagonists which exhibit activity toward both CCK A and CCK B receptors include benzotript and proglumide. Examplary CCK A receptor antagonists include L-364,718 (devazepide); loxiglumide; dexloxiglumide; lorglumide; L-lorglumide; D-lorglumide; PD-140,548; TP-680; T-0632; A-67396; A-70276; A-71134 and SR 27897. Examplary CCK B receptor antagonists include CR2945; YMO22; itriglumide; L-740,093; L-365,260; L-156,586; LY-262691; ureidoacetamides (e.g., RP 69758, RP 72540, RP 73870); tetronothiodin; peptide analogs (CI-1015 and CI-988); YF476; A-63387 and GV150013X. Other examplary CCK receptor antagonists include, but are not limited to, A-64718; A-65186; spiroglumide; CR-2345; CR-2767; CR2622; tarazepide; L-365,260; L-708,474; L-368,730; L-369,466; L-736,380; FK-480; FR175985; FR193108; FR196979; FR202893, FR208418; FR208419; CP212,454; CP310,713; GV191869X; GV199114X; RPR1011367; S-0509; DA-3934; D51-9927; LY-202769; CCK-8; CCK-4; CAMI 189; PD-135,666; CAM1481; PD-140,547; PD-140,723; PD-149,164; JB93182; AG-041R; SR-27,897 (linitript); KSG-504; and 2-NAP.

The present medicaments can optionally be combined with one or more NSAIDs, e.g., aspirin, choline and magnesium salicylates, choline salicylate, celecoxib, diclofenac potassium, diclofenac sodium, diclofenac sodium with misoprostol, diflunisal, etodolac, fenoprofen calcium, flurbiprofen, ibuprofen, indomethacin, ketoprofen; magnesium salicylate, meclofenamate sodium, mefenamic acid, meloxicam, nabumetone, naproxen, naproxen sodium, oxaprozin, piroxicam, rofecoxib, salsalate, sodium salicylate, sulindac, tolmetin sodium, or valdecoxib.

The present medicaments can optionally be combined with one or more prostanoid receptor antagonists, e.g., AH-6809, ONO-8711 and ONO-8713, L-161,982 (4), AH-23848 (4), ONO-AE3-208 (4), SC19220, ONO-8711, SC51089, L-798,106, prostanoid receptor antisense oligonucleotides, ONO-8713, ONO-AE829, ONO-AE2-227, SC51322, ZD-6416, ONO-3144, ONO-3708, ONO-NT-12, SC-236, SC-299, SC-51234A, SKF-104493, SKF-105561, SKF-105809, SKF-106978, SKF-86002, SQ-28852, CGP-47969A, CGS-11776, FR-122047, L-640035, L-651392, L-651896, L-655240, L-657925, L-657926, L-745296, L-745337, L-746483, L-761066, L-768277, PD-138387, WY-48090, S-5751, BWA868C, ramatroban, Ifetroban, SQ-29548, ICI-192605, S-1452, FCE-27262, ONO-8809, ONO-NT-126, CBS-1108, L-636499, diphloretin phosphate, polyphloretin phosphate, BM-613, BM-500, BM-519, BM-567, BM-573, ONO-3708, seratrodast, GW627368 and taprostene, YM-158, YM-26734, YM-57158, Z-335, ZD-9583, 6-[N-(2-benzyloxy-5-bromobenzyl)-N-ethylamino]pyridazine-3-carboxylic acid, 6-[N-(5-bromo-2-(2-methylprop-2-en-1-yloxy) benzyl)-N-ethylamino]pyridazine-3-carboxylic acid (disclosed in Example 15 of International Patent Publication No. WO 97/00864), N-propanesulphonyl-6-[N-(5-bromo-2-(cyclopropylmethoxy)benzyl)-N-ethylamino]pyridazine-3-carboxamide (disclosed in Example 14 of International Patent Publication No. WO 97/00863), N-(3,5-dimethylisoxazol-4-ylsulphonyl)-6-[N-(5-chloro-2-(2-m ethylpropoxy)benzyl-N-ethylamino]pyridazine-3-carboxamide (disclosed as compound number 1 in Example 8 of International Patent Publication No. WO 97/00863), 6-[N-(5-bromo-2-(cyclopropylmethoxy)benzyl)-N-ethylamino]pyridazine-3-carboxylic acid (disclosed in Example 3 of International Patent Publication No. 97/00863), and the like.

The present medicaments can optionally be combined with one or more CGRP receptor antagonists, e.g., BIBN4096BS, SB-(+)-273779, CGRP₈₋₃₇, Compound 1 (4-(2-oxo-2,3-dihydro-benzoimidazol-1-ŷ-piperidine-1-carboxylic acid [1-(3,5-dibromo-4-hydroxy-benzyl)-2-oxo-2-(4-phenyl-piperazin-1-yl)-ethyl]-amide), and other CGRP receptor antagonists (see, Arulmani et ah, 2004, Eur J Pharmacol 500:315-330 for review)

The present medicaments can optionally be combined with one or more opioids, e.g. buprenorphine, butorphanol, codeine, fentanyl, hydrocodone, hydromorphone, methadone, morphine, oxycodone, or propoxyphene.

The present medicaments can optionally be combined with one or more glutamate antagonists, e.g. NMDA antagonists, AMPA antagonists, or kainate receptor antagonists. Examplary glutamate receptor antagonists include D-AP5 (D(−)-2-amino-5-phosphonopentanoate), CGS19755 (4-phosphonomethyl-2-piperidine carboxylic acid), CGP37849 (D,L-(E)-2-amino-4-methylphosphono-3-pentanoic acid), LY233053 (cis-(±)-4-(2H-tetrazol-5-yl)methyl-piperidine-2-carboxylic acid), AIDA (1-aminoindan-1,5(RS)-dicarboxylic acid), (S)-(+)-CBPG ((s)-(+)-2-(3′-carboxy-bicyclo(1.1.1.)pentyl)glycine), CPCCOEt (cyclopropan(b)chromen-1a-carboxylate), EGLU ((s)-(a)-ethylglutamate), LY307452 (2s,4s-2-amino-4-(4,4-diphenylbut-1-yl)pentan-1,5-dioc acid) LY341495 (2s-2-amino-2-(1s,2s-2-carboxy-cyclopropan-1-yl)-3-(xanth-9-yl)propanoic acid), PCCG-4 (2s,1′s,2′s,3′R)-2-(2′-carboxy-3′-phenylcyclopropyl)glycine), 4-CPG (4-carboxyphenylglycine), memantine, and amantadine.

The present medicaments can optionally be combined with one or more NMDA antagonist, e.g., L-glutamate derivatives, tetrahydroquinoline, imidazoloquinoxalinone, isatine, fused cycloalkylquinoxalinediones, quinoxaline, spermine, a 4-hydroxy-3-nitro-1,2-dihydroquinolon-2-one derivative, an indole derivative, a benzo-thiadiazine dioxide derivative, an indeno(1,2-b)pyrazin-3-one or corresponding 2,3-dione, a quinoline derivative, an ethyl (phenylcarbamoyl)ethenyl)dichloroindole carboxylate, a thienopyrazine 2,3-dione derivative, a 2-(2,3-dicarboxycyclopropyl) glycine, a 2-amino-3-substituted phenyl propionic acid derivative, 1-carboxyalkylquinoxaline-2,3(1H,4H) dione derivative, a thienyl-glycine derivative, a benzo-fused azacyclic compounds, an indole derivatives, a tricyclic quinoxaline-diene derivative, a 3-hydroxy anthranilic acid and salts, a decahydroisoquinoline compound, a tri- or tetra-substituted guanidine derivatives, a D- or L-tryptophan derivative, a tetrazolyl(alkyl)-cyclohexykaminoacid derivative, an octahydrophenanthrene derivative, a benzomorphan compound, a piperazinyl or piperidinyl-alkyl substituted isoxazole derivative, a decahydroisoquinoline-3-carboxylic ester or amide preparation, a compounds based on Conantokin-G peptide, a 3-heterocyclykalkyl-benzopyran-2-orie derivative, a phosphono-alkyl imidazo-pyrimidine carboxylic acid derivative, amantidine, memantine, rimantidine, a histogranin peptide or analogue, a nitrobenzoic acid derivative, e.g. 44(2-methoxycarbonyl-4-nitrophenyl)methyl) piperazine carboxylic acid, a diamine derivative with selective sigma receptor affinity, remacemide (2-amino-N-(1,2-diphenyl-1-methylethyl)acetamide), a phosphono-alkylidene- or phosphono-alkoxyimino-piperidine acid, a benzothiadiazine carboxylic acid derivative, a dihydro-benzothiadiazine dioxide carboxylic acid derivative, a 4-hydroxy 2 (H) pyrrolone derivative, a quinoxaline derivative, a tetrahydro-imidazo (1,2-a) pyrimidines or its salt, a alpha-amino acid, a 4-hydroxy-pyrrolo[1,2-b]pyridazin-2(1H)-one derivative, a nitroquinolone derivative, a 3-aryl-substd 2(1H)quinolone, a 2(1H)-quinolone, a phosphonic acid quinoline-2-carboxylic acid derivative, its per hydro quinoline derivative or salt, a benzimidazole(s) carrying 2 acidic groups, an N,N′-disubstituted guanidine derivative, a tricyclic quinoxaline dione, a 2-(2,3-dicarboxycyclopropyl) glycine stereoisomer, pregnenolone sulphate or one of its derivative, an isatine derivative, a 3-amino-indolyl-derivative, 2-phenyl-1,3-propanediol dicarbamate (felbamate), a benzomorphan derivative, a dihydrothienopyridine derivative, an enantiomer of (aminophenyl)-heteroaryl ethylamine, a pyridazine-dione derivative, a 2H-1-benzopyran-2-one compound, a 4-sulphonylamino-quinoline derivative, a R(plusy3-amino-1-hydroxy-pyrrolidine-2-one, a 2-carboxy indole, a substd. imino-methano dibenzo (A,D) cycloheptene derivative, an indole-hydrazone, a piperazine derivative, a 4,6-disubstituted tryptophan and kynurenine derivative, a fluorenamine compound, a diketo-pyrido pyrazine derivative or its salts, a 2-amino-3,4-dioxo-1-cyclobutene derivative, a 2-acyl-amido derivative of 3,4-dihydro-3-oxo-quinoxaline, a benzimidazole phosphono-aminoacid derivative, a quinoxaline phosphono-aminoacid derivative, a piperazine, piperidine or pyrrolidone derivative, ist salts and isomeric forms including stercoisomers, δ 4-hydroxy-2(1H)-quinolinone derivative, ist salts and prodrugs, a fused pyrazine derivative, a 2-phenyl or 2-thienyl-(2)-piperidine derivative, a 3-amido or 3-sulphamido-indolyl derivative, a 3-aryl-4-hydroxy-2-(1H)-quinolone derivative, a 2-heterocyclyk2-hydroxy-ethylamine derivative, a 1-aryl-2-aminomethyl pyrrolidine, its optical isomers and acid-addn. salts, a 4,6-dihalo indole2-carboxylic acid derivative, a cyclic aminohydroxamate derivative, a tetracyclic amine derivative, a 2,4-dioxo-1,2,3,4-tetrahydroquinoline derivative, a 2,4-dioxo-1,2,3,4-tetrahydroquinoline derivative, a 3-phosphonopiperidine and p-pyrrolidine derivative, a benzothieno (2,3-B)-pyrazine-2,3-(1H,4H)-dione, a spiro dibenzosuberane derivative, a benzomorphan derivative, a preparation of 3,4-disubstituted 2-isoxazoline(s) and isoxazoles(s), a 3-indolyl thio-acetate derivative, an arginine-derived nitric oxide biosynthesis inhibitor, a dicyclic amine derivative, a spiroisoindole derivative, an imidazo(1,2-A)-pyridinylalkyl compound, a 1,2,3,4-tetrahydro-9H-pyrido indole or benzothiophene derivative, an indole-2,3-dione-3-oxime derivative, a 1-aryl-2-(aminomethyl) cyclopropanecarboxamide derivative, a 4-phosphono-2-amino-alkenoic acid derivative, a naphthopyran derivative, a beta-ketone, a beta oxime or beta hydrazine phosphonate, a topa quinone aminoacid, kynurenic acid or a derivative, a quinoline- or thienopyridine-carboxylic acid derivative, a 10,5^{˜}(imino-methano)-10,11-dihydro-5H-dibenzo(A,D)cyclohept ene or a derivative, a bicyclic amino-hydroxamate derivative, an indole-2-carboxylic acid derivative, a substituted adamantane derivative, a benzobicycloalkane derivative, a 2,4-disubstituted-1,2,3,4-tetrahydro-quinoline derivative, a dihydro-alkyl-substituted (immunomethano)-5H-dibenzo-cycloheptene, an aryl cyclohexylamine, an N-substd. benzobicycloalkane amine, an isoquinoline phosphonate derivative, an N,N′-disubstd.-guanidine compound, a phosphonopropenyl piperidine carboxylic acid compound, (2R,3S,4S)-alpha-carboxycyclo-propyl-glycine, a pyrrolidine derivative, a dihydroxy-fused heterocyclyl quinoxaline derivative, a hydrogenated derivative of MK801 and analogues, a 5-substd. 10,11-dihydro 5H-dibenzo (A,D) cycloheptene 5,10-imine, an 11-Exo-hydroxy MK 801 preparation including electrochemical cyclisation step to form 5,10-imine bridge in 5-methyl 5-oxyamino 5H-dibenzo (A,D) cycloheptene, a tetrahydro-isoquinoline or 2-benzazepine derivative, an N-3-phenylpropionyl-substd. spermine or related polyamine derivative, a 4α-amino-fluorene compound or a heterocyclic analogue, a cyclooctane-imine derivative, a R-3-amino-1-hydroxy pyrrolidin-2-one or methionine hydroxamate, a 10,11-dihydro-5H-dibenzo-cyclohepten-5,10-imine compound, a polyhydro-10,11-dihydro-5H-benzo(a,d)cyclohepten-5,10 imine derivative, a 4-oxo-1,4-dihydroquinoline compound with 2-acidic groups, a heterocyclykalkene-phosphonic acid compound, a phosphono gp-containing pyridine 2-carboxylic acid, an alpha-amino-alpha-(3-alkylphenyl)alkyl ethanoic acid, its esters or amides, a 10,11-dihydro-5H-dibenzo-A,D-cyclohepten-5,10-imine compound, a phosphorus containing unsaturated amino acid or its salts, a 5 Substd.-1-, 11-dihydro-5H-dibenzo-cyclohepten-5,10-imine or analogue, a heterocyclic phosphonic acid derivative or its salt, a substituted 4-(aminocarbonyl-amino)quinoline derivative, a tricyclic quinoxaline derivative, a butyryltyrosinc spermine or one of its analogue, a tri- or tetra-substituted guanidine, a quinoxalinylalkyl-aminoalkane phosphonic acid derivative, a 2-(aminophenyl)-3-(2-carboxy-indol-3-ylypropenoic acid derivative, a 6-piperidinylpropiony-2(3H)-benzoxazolone derivative, 6-(3-[4-(4-fluorobenzyl)piperidin-1-yl]propionyl)-3H-benzoxazol-2-one or one of its salts, an imidazo[1,2-a]pyridine compound, a tetrahydroquinoline derivative or one of its salts, a 2-methyl-5,8-substituted 2,3,4,5-tetra- or 2,3A4a,5,9b-hexahydro-1H-pyrido[4,3-b]indole, a 3-aminoindolyl compound, a 6-pyrrolyl-quinoxaline-2,3-dione derivative, an imidazoly(mercaptoalkyl)-quinoxaline-dione compound, a 3-amidoindolyl derivative, a heterocyclyl-imidazolo-quinoxalinone compound, a naphthyl-substituted alpha-amino acid derivative, a 5-heteroaryl-2,3-quinoxaline-dione derivative, a quinoxaline derivative, a 5H, 10H-imidazo indeno 4-pyrazinone derivative, a hydroxy-(aryl-substituted phenyl)-quinolone compound, an imidazo indolo pyrazinone derivative, a ((phenyl-amino)-(m) ethylypyridine derivative, a tetrahydro-isoquinoline derivative, a 4-substituted piperidine analogue, a 2-substituted piperidine derivative, a tri- or tetra-substituted guanidine derivative, a 3-Hydroxy-4-imidazolidinone, a 3-aminoquinoxalin-2-one derivative, rapamycin or a derivative e.g. 1,3-Diels Alder adduct with phenyl-triazoline-dione, 1-amino-1-cyclobutanecarboxylic acid, a thiamorphinan derivative, a pyrido[4,3-b]indole derivative, 4-phenyl carbamoyl methylene tetrahydro quinoline-2-carboxylic acid or a derivative thereof, (3R,4SY3-(4-(4-fluorophenyl)-4-hydroxy-piperidin-1-yl)-chroman-4,7-dioaphenol derivative, an indeno-pyrazin-4-one, a 2,3-dioxo-1,2,4,5-tetrahydro-quinoxalinyl derivative, a 45-bridged quinoxalinedione or quinolone, (1S,2S)-1-(4-hydroxyphenyl)-2-(4-hydroxy 4-phenyl piperidin-1-yl) I-propanol methane sulphonate trihydrate, a 4-sulphanimide-quinoline derivative, a methanobenzocyclodecen-13-amine compound, a derivatives of pregnenolone sulphate, a quinoxalinyl-(alkane,alkene, or alkyne)-phosphonic acid or one of ist esters, a diarylalkylamine related to spider and wasp venom toxins, a piperazine R-alpha-carboxylic acid derivative, an imidazo-indeno-pyrazin-4-one derivative, a pyridazino-quinoline derivative, a 1-substituted, or 1,3-di-substituted, 1,3-diaryl-guanidine compound, an aza-cycloalkykfused quinoxaline-dione, a 3-substd, 2-carboxy-indole derivative or intermediate, a (2R)-N-trityl-4-oxo-5-(dimethyl phosphono)-nor-valinate ester, a kynurenic acid derivative, an indole carboxylic acid derivative, a 6-(tetrazolyl or isoxazolyl-decahydroisoquinoline-3-carboxylic acid derivative, a phenyl- or pyridinyl-thieno-pyridinone derivative, a fused cycloalkylquinoxaline-dione derivative, a pyridazino-quinoline derivative, a 1-Alpha-amino-3-biphenyl-propanoic acid derivative, a 3-(Indol-3-yl) propenoic acid derivative, a spiro-heterocyclemidazo-indeno-pyrazine-4-one derivative, a 2-heterocyclyk3-indolylpropenoic acid derivative, a piperidinoalkyl heterocyclic ketone or alcohol compound, a pyrrolyl-tetrahydro-benzoquinoxaline-dione derivative, a 7-imidazolyl or dialkylamino, tetrahydroquinoxaline dione compound, a dibenzocycloheptene, a quinoxaline derivative, an aryl-thio-quinoxaline derivative, a heterocyclic substd. imidazolo-quinoxaline derivative, a 1,4-dihydro-quinoxaline-2,3-dione derivative, an oxa- or thia-aliphatically bridged quinoxaline derivative, an aza-aliphatically bridged quinoxaline-2,3-dione compound, a 3-amido- or 3-sulphamido-indole compound, a 3,5-disubstd. phenyl-naphthalene derivative, an imidazo (1,2-a)indeno (1,2-e) pyrazine-2-carboxylic acid derivative, a 3-phenyl-fused ring pyridine-dione derivative, a 2-phenyl-pyridazino-indole-dione derivative, a 4,6-disubstd. kynurenine compound, a phosphono derivative of imidazo(1,2-a)pyrimidine-2-carboxamide, a tetrahydro-quinoxaline-dione derivative with N-(alkyl)carbonyl-amino- or ureido group, a tryptophan derivative, a hetero-aliphatic or hetero-araliphatic substd. quinolone derivative, an imidazo-pyridine dicarboxylic acid derivative, a composition containing pyrazolo-quinoline derivatives, an ethanodihydrobenzoquinolizinium salt, an oxopyridinylquinoxaline derivative, an indeno-triazolo-pyrazin-4-one derivative, an imidazo-indeno-pyrazinone derivative, an imidazo-indeno-pyrazin-4-one derivative, an imidazo[1,2-a]pyrazine-4-one derivative, a 5H-indeno-pyrazine-2,3-dione derivative, a phenyl-aminoalkyl-cyclopropane N,N-diethyl carboxamide compound, a dexanabinol derivative, a substituted chroman derivative, a sulphonamide quinazoline-2-4-dione compound, a 6- and 8-aza-, and 6,8-diaza-1,4-dihydro-quinoxaline-2,3-dione derivative, a substituted quinoline derivative, a tetrazolylalkyl cyclohexyl aminoalkanoic acid, a tricyclic indole 2-carboxylic acid derivative, a 6-substd-7H-imidazo-8-pyrazinone derivative, a quinoxaline dione derivative or one of its radiolabelled compounds, a tricyclic pyridazinopyridine derivative, an N-substituted heterocyclylidenemethyl-indole carboxylic acid derivative, a 3-aza-8-substituted-bicyclo(3.3.0)octane-2-carboxylic acid derivative, an ethano-heterocyclo-isoquinolinium salt, a phenyl alkanolamine derivative, a dihydrobenzothiadiazinedioxide carboxylic acid derivative, a methyl-butenylmethyl(hydroxy-propyl)carbazoledione, an imidazo pyrazinone derivative, an imidazo-(1,2-a)pyrazine-4-one, a benzazepine-dione derivative, disulfuram, a 3-(indol3-yl)-propenoic acid derivative, a 1,2,3,4-tetrahydro-quinoline-2,3,4-trione-3 or 4-oxime compound, a peptide antagonist at NMDA receptors, a 2-amino-2-phenyl(alkylyacetic acid derivative, 6-halo-tryptophan or a 4-halo-kynurenine, a 6-tetrazolyl orisoxazoly-decahydro-isoquinoline-3-carboxylic acid derivative, an an imidazolylbenzene or salts thereof.

The present medicaments can optionally be combined with one or more AMPA antagonists, e.g., L-glutamate derivatives, amino alkanoic acid derivatives, α-amino-3-hydroxy-5-methyl-4-isoxazolepropionate derivatives, acetyl-aminophenyl-dihydro-methyl-methyl-dioxolobenzodiazepine, acid amide derivatives, amino-phenyl-acetic acid, 2,3-benzodiazepin-4-one, alkoxy-phenyl-benzodiazepine, amino- or desamino 2,3-benzodiazepine, benzothiadiazine, α-carboline-3-carboxylic acid, fused cycloalkylquinoxalinediones, decahydroisoquinoline, 4-hydroxypyrrolone, 4-hydroxy-pyrrolo-pyridazinone, imidazo-pyrazinone, imidazolo-quinoxalinone, indeno-pyrazine-carboxylic acid, indeno-pyrazinone, indoloneoxime, indolo-pyrazinone, isatine, isatinoxime, oxadiazole, phenyl-azolophthalazine, phenylpyridazino-indole-1,4-dione, quinoline, quinolinone, quinoxaline, quinoxalinedione, quinazolinone, quinolone, nitroquinolone, and sulphamate derivatives.

The present medicaments can optionally be combined with one or more kainate receptor antagonists, e.g., L-glutamate derivatives, kainic acid derivatives, acid amide derivatives, aminoalkanoic acid derivatives, aminophenyl(alkyl)acetic acid derivatives, fused cycloalkylquinoxalinediones, quinoxalinedione, imidazolo-quinoxalinone, isatine, phenylazolophthalazine, pyridothiazines, 4-phosphonoalkyl-quinolinone, quinolinone, quinazoline, quinazolinedione, quinoxalinedione, and sulphamate derivatives.

The present medicaments can optionally be combined with one or more inhibitors of glutamate release, e.g., lamotrigine, BW1003C87, riluzole, isoguvacine, muscimol, THIP, piperidine-4-sulphonic acid, flunitrazepam, zolpidem, abecamil, ZK93423, L-baclofen, CGP27492, piracetam, progabide, and CGP35024.

The present medicaments can optionally be combined with one or more glutamate reuptake promoters, e.g. zonisamide.

The present medicaments can optionally be combined with one or more combined serotonin/noradrenaline reuptake inhibitors, e.g., venlafaxine, milnacipran, duloxetine, pregabalin, LY248686, and Strattera.

The present medicaments can optionally be combined with one or more tricyclic antidepressants, e.g., amitriptyline, amitriptylinoxide, butriptyline, clomipramine, demexiptiline, desipramine, dibenzepin, dimetacrine, dosulepin/dothiepin, doxepin, imipramine, imipraminoxide, lofepramine, melitracen, metapramine, nitroxazepine, nortriptyline, noxiptiline, pipofezine, propizepine, protriptyline, and quinupramine.

The present medicaments can optionally be combined with one or more tetracylic antidepressants, e.g., amitriptyline, amitriptylinoxide, butriptyline, clomipramine, demexiptiline, desipramine, dibenzepin, dimetacrine, dosulepin/dothiepin, doxepin, imipramine, imipraminoxide, lofepramine, melitracen, metapramine, nitroxazepine, nortriptyline, noxiptiline, pipofezine, propizepine, protriptyline, or quinupramine.

The present medicaments can optionally be combined with one or more dopamine antagonist, e.g., domperidone, droperidol, haloperidol, chlorpromazine, promethazine, prochlorperazine, alizapride, or prochlorperazine.

The present medicaments can optionally be combined with one or more nk1 receptor antagonists, e.g., aprepitant or casopitant.

The present medicaments can optionally be combined with one or more antihistamine, e.g., cyclizine, diphenhydramine dimenhydrinate, meclizine, promethazine, or hydroxyzine.

The present medicaments can optionally be combined with one or more benzodiazepines, e.g., midazolam or lorazepam.

The present medicaments can optionally be combined with one or more anticholinergics, e.g., hyoscine.

The present medicaments can optionally be combined with one or more or steroids, e.g, dexamethasone.

Examplary Medicaments

By way of example, any of the formulations set forth in Table 1 can usefully be used with the treatment methods of the present invention.

TABLE 1
Examplary Medicaments

| | Total Cannabinoid | |
|---|---|---|
| formulation # | amount (mg) | Dosage Compartment |
| 1 | 0.1 | oil |
| 2 | 0.25 | oil |
| 3 | .5 | oil |
| 4 | 1 | oil |
| 5 | 2 | oil |
| 6 | 0.1 | oil |
| 7 | 0.25 | oil |
| 8 | .5 | oil |
| 9 | 1 | oil |
| 10 | 2 | oil |
| 11 | 0.1 | oil |
| 12 | 0.25 | oil |
| 13 | .5 | oil |
| 14 | 1 | oil |
| 15 | 2 | oil |
| 16 | 0.1 | oil |
| 17 | 0.25 | oil |
| 18 | .5 | oil |
| 19 | 1 | oil |
| 20 | 2 | oil |
| 21 | 0.1 | crystalline form |
| 22 | 0.25 | crystalline form |
| 23 | .5 | crystalline form |
| 24 | 1 | crystalline form |
| 25 | 2 | crystalline form |
| 26 | 0.1 | SEDDS IR |
| 27 | 0.25 | SEDDS IR |
| 28 | .5 | SEDDS IR |
| 29 | 1 | SEDDS IR |
| 30 | 2 | SEDDS IR |
| 31 | 0.1 | crystalline form |
| 32 | 0.25 | crystalline form |
| 33 | .5 | crystalline form |
| 34 | 1 | crystalline form |
| 35 | 2 | crystalline form |
| 36 | 0.1 | oil |
| 37 | 0.25 | oil |
| 38 | .5 | oil |
| 39 | 1 | oil |
| 40 | 2 | oil |
| 41 | 0.1 | aqueous liquid |
| 42 | 0.25 | aqueous liquid |
| 43 | .5 | aqueous liquid |
| 44 | 1 | aqueous liquid |
| 45 | 2 | aqueous liquid |
| 46 | 0.1 | oil |
| 47 | 0.25 | oil |
| 48 | .5 | oil |
| 49 | 1 | oil |
| 50 | 2 | oil |
| 51 | 0.1 | aqueous liquid |
| 52 | 0.25 | aqueous liquid |
| 53 | .5 | aqueous liquid |
| 54 | 1 | aqueous liquid |
| 55 | 2 | aqueous liquid |
| 56 | 0.1 | Adsorbate solid |
| 57 | 0.25 | Adsorbate solid |
| 58 | .5 | Adsorbate solid |
| 59 | 1 | Adsorbate solid |
| 60 | 2 | Adsorbate solid |
| 61 | 0.1 | Complexation Solid |
| 62 | 0.25 | Complexation Solid |
| 63 | .5 | Complexation Solid |
| 64 | 1 | Complexation Solid |
| 65 | 2 | Complexation Solid |
| 66 | 0.1 | Adsorbate solid |
| 67 | 0.25 | Adsorbate solid |
| 68 | .5 | Adsorbate solid |
| 69 | 1 | Adsorbate solid |
| 70 | 2 | Adsorbate solid |
| 71 | 0.1 | Complexation Solid |
| 72 | 0.25 | Complexation Solid |
| 73 | .5 | Complexation Solid |
| 74 | 1 | Complexation Solid |
| 75 | 2 | Complexation Solid |

In another embodiment, a medicament comprises a formulation comprising the components set forth in Table 1. The dosage compartment can be formulated, for example, as an IR dosage compartment.

In one embodiment, a medicament comprises a formulation comprising components set forth in Table 1, except that the total cannabinoid amount is +/−50% (e.g. +/−30% or +/−20%) of that listed in Table 1.

Therapeutic Window

Medicaments of the present invention provide a useful therapeutic window. According to the present invention, a useful therapeutic window is about 6 to about 12 hours, about 6 to about 9 hours, about 6 to about 8 hours, about 7 to about 8 hours, or about 7 to about 9 hours.

Optionally, within an hour or two following the end of the therapeutic window, the plasma levels are reduced below a level that can result in undesired side effects such as impeded cognitive and/or psychomotor performance, tachycardia, hypotension, or impaired learning and memory.

Dosing and Titration

The skilled artisan will recognize (with the teachings of this invention) that “optimal” dosing of an individual is determined by evaluating, among other factors, efficacy and safety (i.e. the “therapeutic profile”). As demonstrated in Example 6, the inventors have surprisingly observed that subjects with certain cannabinoid-sensitive disorders can demonstrate an increased-responsiveness to medicaments of the present invention upon a prolonged treatment period (e.g. about one week or longer, or about 2 weeks or longer, or about one month or longer, or about six months or longer).

One embodiment of the present invention is a method for treating cannabinoid-sensitive disorders which comprises initially administering a cannabinoid dose for a treatment period followed by administering a lower dose of a cannabinoid. As taught herein, this method can provide therapeutic efficacy during each treatment period and yet reduces the total drug load. In another embodiment, the method comprises administering a sub-optimal cannabinoid dose and continuing such treatment for a prolonged treatment period and then evaluating efficacy and side effects before modifying the dose.

In another embodiment of the present invention, a cannabinoid-sensitive subject is titrated as taught here to determine optimal dose. Subjects are initially administered a low dose of the medicament for a treatment period. At the completion of the treatment period, the dose in the medicament is increased or the number of medicament units is increased (a “step-up”) for an additional treatment period. This “escalation” cycle can be repeated multiple times until 1) optimal clinical benefit is achieved, 2) clinically relevant side effects become apparent, or 3) until the maximum dose generally considered safe is administered.

Optionally, treatment-related side effects (or trial related adverse events) are evaluated during treatment periods. Relevant evaluations include mental alertness, emotional health, quality of life, sleepiness, etc.

Optionally, a clinically-relevant metric(s) of the disorder or condition being treated is assessed during each treatment period. Methods are readily known for quantifying or assessing apnea, pain, spasms, etc.

Optionally, an initial “low dose” THC medicament of the present invention can contain about any mg amounts of a cannabinoid, e.g. 0.1, 0.5, 1, 2, 5, 10, 20, or 50 (mg).

Optionally, a treatment period for each dose is about 1 to about 10 days or about 5 to about 10 days, or longer than 10 days. Administrations can be provided, for example, daily, multiple times per day, or 2-7 times per week.

A typical step-up dose increase is any percent of about 10, 20, 25, 33, 50, 100, 200, or 400(%).

As a result of the titration study, an empirically determined dose that is well tolerated (minimal or no significant side effects) and optimally effective is selected. This selected dose is administered for another period (e.g. 1 or more days or more than 1 week or more than 1 month.

Optionally, the subject is administered a “step down” lower dose medicament (e.g. about 50% to about 75% or about 20% to about 50% of the previous dose). A clinically relevant metric of efficacy and side effects are assessed. If therapeutic efficacy is not diminished (over the previous dose), the subject can optionally be administered a dose with a further reduction (i.e. a second or subsequent step-down).

Optionally, the subject has a sleep apnea and is administered a medicament comprising an amount of a THC in the range of about 0.05 mg to about 5 mg (e.g. administered 0.5, 1, 1.5, or 2 hrs before anticipated sleep time or sleep cycle) for a treatment period (e.g. about 5 to about 30 days). During this treatment period, overnight PSG is optionally performed. If the patient tolerates this dose (e.g. minimal treatment related side effects), a step-up dose is administered daily for another treatment period. Therapeutic profile is assessed, and a subsequent escalation is performed until clinically-relevant side effects are observed or maximal safe dose is administered.

A step-down titration is optionally performed and evaluated.

In one embodiment of the present invention, a kit is provided that contains an appropriate number of one or more doses of a medicament (otherwise of the same formulation). The kit optionally contains patient instructions. Optionally, the doses are in a device that compartmentalizes the daily doses (e.g. a blisterpack).

Safety

Unexpectedly, it has been discovered that oral administration examplary present medicaments maintains a therapeutic window while not resulting in a plasma levels at any time throughout the treatment window (sleep period) that increase the likelihood of side effects. Such side effect-sparing medicaments avoid one or more of the effects shown in Table 2.

Unexpectedly, it has been discovered that oral administration of examplary present medicaments maintains a therapeutic window (sleep period) while not resulting in a plasma levels at any time throughout the treatment window that increase the likelihood of side effects associated with co-administration of other prescription or over the counter medicines such as shown in Table 3.

TABLE 2
Dronabinol Side Effects

| | Side Effects |
|---|---|
| Cardiovascular: | Conjunctivitis*, hypotension*., |
| | tachycardia, hypotension |
| Digestive: | Diarrhea*, fecal incontinence. |
| Musculoskeletal: | Myalgias. |
| Nervous system: | Depression, nightmares, speech |
| | difficulties, tinnitus. impeded |
| | cognitive and/or psychomotor |
| | performance,, or impaired learning |
| | and memory |
| Skin and Appendages: | Flushing*. |
| Special senses: | Vision difficulties. |

*Incidence of events 0.3% to 1%

TABLE 3
Dronabinol Side Effects Associated with Co-administration of Other Drugs

| Drug | Side effects |
|---|---|
| Amphetamines, cocaine, other | Additive hypertension, tachycardia, possibly |
| sympathomimetic agents | cardiotoxicity |
| Atropine, scopolamine, | other anticholinergic agents, Additive or super- |
| antihistamines, | additive tachycardia, drowsiness |
| Amitriptyline, amoxapine, | Additive tachycardia, hypertension, drowsiness |
| desipramine, other tricyclic | |
| antidepressants | |
| Barbiturates, benzodiazepines, | Additive drowsiness and CNS depression |
| ethanol, lithium, opioids, buspirone, | |
| antihistamines, muscle relaxants, | |
| other CNS depressants | |
| Disulfiram A | reversible hypomanic reaction was reported in a 28 |
| | y/o man who smoked marijuana; confirmed by |
| | dechallenge and rechallenge |
| Fluoxetine | A 21 y/o female with depression and bulimia |
| | receiving 20 mg/day fluoxetine X 4 wks became |
| | hypomanic after smoking marijuana; symptoms |
| | resolved after 4 days |
| Antipyrine, barbiturates | Decreased clearance of these agents, presumably |
| | via competitive inhibition of metabolism |
| Theophylline | Increased theophylline metabolism reported with |
| | smoking of marijuana; effect |

Methods of Administering a Medicament

In one embodiment, a medicament of the present invention is administered chronically, i.e. a plurality of administrations over a prolonged treatment period such as more than one day, at least a month, or more than one year. As demonstrated in Example 6, upon prolonged treatment; greater efficacy was demonstrated with doses that were initially suboptimal. According, in one embodiment a subject is treated for a prolonged period (e.g. at least 30 days) with an oral cannabinoid in an amount of about 0.05 mg to about 25 mg or optionally about any of the following amounts (in mg): 0.1 to 20, 0.5 to 10, 0.5 to 5, 0.05-2.5, 0.05-2.0, 0.05-1.0, less than 5, less than 2.5, or less than 2.0.

Optionally, the plurality of administrations over the prolonged treatment period is selected from: daily administrations, multiple administrations per day, or 2-7 times per week.

The unexpected results from the clinical studies reported here teach useful doses for populations and for individuals. However, the skilled artisan will readily recognize from these studies that the therapeutic level and side-effect producing levels of plasma THC can vary within individuals. For example, while a 2.5 mg THC medicament of the present invention typically will provide a side-effect sparing efficacy for an extended treatment window, certain individuals will have a higher threshold for both therapeutic efficacy and for side effects. The same is true for certain individuals who will have a lower threshold for efficacy and for side effects. Therefore, there is a remarkable and unexpected utility of the present medicaments containing a cannabinoid in the full range of about 0.05 mg to about 25 mg.

In one embodiment, the invention provides a method of treating apnea comprising administering less than about 20 mg (e.g. less than 10 mg, less than 5 mg, less than 2.5 mg, or 0.05-2 mg) of cannabinoid during a therapeutic window taught herein.

Utility

The present methods and medicaments are useful for treating cannabinoid-sensitive disorders.

The present methods and medicaments are especially useful for treating apnea. Optionally, the apnea is any of obstructive sleep apnea syndrome, obstructive sleep apnea/hypopnea syndrome, upper airway resistance syndrome, apnea of prematurity, congenital central hypoventilation syndrome, obesity hypoventilation syndrome, central sleep apnea syndrome, Cheyne-Stokes respiration, and snoring. Unexpectedly, the present compositions are useful to reduce episodes of apnea, snoring, and sleep disruption, for example as demonstrated by oximetry, or polysomnogram (“PSG”), or self-assessment.

In one embodiment, the administered dose and/or medicament comprises 1 mg to 25 mg.

At least some of the pharmacological effects of THC are exerted through the cannabinoid pathway, by interaction with cannabinoid receptors such as CB1 and/or CB2. Cannabinoid receptors are found predominantly at nerve terminals where they have a role in retrograde regulation of synaptic function and interact with at least acid (e.g. GABA, glutamate), monoamine (e.g. histamine, dopamine, serotonin, noradrenaline) purine (e.g. adenosine, ADP, ATP), peptide (e.g. somatostatin, neuropeptide Y, neurokinin, cholecystokinin), vanilloid, prostanoid, opiod and/or other pathways. For example, THCs such as delta-9-tetrahydrocannabinol act as agonists at CB1 and CB2 receptors, mimicking the effects of the naturally occurring endocannabinoids, which modulate the effects of neurotransmitters. Without being bound by theory, the inventors believe that the methods and medicaments of the present invention exert pharmacological action through modulation of one or more of these pathways. Indeed, the cannabinoid receptors are concentrated in regions of the brain that control functions associated with certain pharmacological effects of cannabinoid modulation (see Table 4).

TABLE 4
Brain Regions in Which Cannabinoid Receptors Are Abundant

| | Examplary Functions Associated |
|---|---|
| Brain Region | with Region |
| Basal ganglia | Movement control |
| Substantia nigra pars reticulata | Movement control |
| Entopeduncular nucleus | Movement control |
| Globus pallidus | Movement control |
| Putamen | Movement control |
| Cerebellum | Body movement coordination |
| Hippocampus | Learning and memory, stress |
| Cerebral cortex, especially | Higher cognitive functions |
| cingulate, | |
| frontal, and parietal regions | Higher cognitive functions |
| Nucleus accumbens | Reward center |
| Hypothalamus | Body housekeeping functions (body |
| | temperature regulation, salt and water |
| | balance, reproductive function) |
| Amygdala | Emotional response, fear |
| Spinal cord | Peripheral sensation, including pain |
| Brain stem | Sleep and arousal, temperature |
| | regulation, motor control |
| Central gray | Analgesia |
| Nucleus of the solitary tract | Visceral sensation, nausea and |
| | vomiting |

Based-upon the insight of the inventors, the present methods and medicaments are surprisingly effective in treating disorders associated with the above-mentioned pathways, brain regions, or pharmacological effects, and other cannabinoid-sensitive disorders. Accordingly, the invention provides a method for treating a cannabinoid-sensitive disorder in a subject comprising administering to the subject a medicament taught herein. Optionally, the cannabinoid-sensitive disorder is a neurological disorder, pain, an appetite or wasting disorder, nausea, vomiting, a seizure disorder, a sleep disorder, breathing disorder, or a sleep-related breathing disorder. Optionally, the method further comprises administering an additional therapeutic agent for treating the disorder. Optionally, an additional therapeutic agent is included in the medicament. Optionally, the additional therapeutic agent is administered sequentially with the medicament.

In one embodiment, the present methods and medicaments are useful for treating a neurological disorder. Optionally, the neurological disorder is of the brain, spinal cord, peripheral nerves, or muscles. Optionally, the neurological disorder is a neurodegenerative disease, a neurological pain, a movement disorder, or a mood disorder.

In one embodiment, the present methods and medicaments are useful for treating a neurodegenerative disease. Optionally, the neurodegenerative disease is multiple sclerosis, Huntington's disease, or Alzheimer's disease.

In one embodiment, the present methods and medicaments are useful for treating a neurological pain. Optionally, the neurological pain is a central or peripheral neurological pain. Optionally, the neurological pain is chronic pain. Optionally, the neurological pain is associated with fibromyalgia, multiple sclerosis, spinal cord injury, or stroke.

In one embodiment, the present methods and medicaments are useful for treating a movement disorder. Optionally, the movement disorder is caused by abnormalities in the basal ganglia. Optionally, the movement disorder is a spasm disorder, muscle spasticity, seizure disorder, chorea, Huntington's disease, dystonia, basal ganglia movement disorder, Parkinson's disease, Tourette's syndrome, dyskinesia, bradykinesia, or epilepsy.

In one embodiment, the present methods and medicaments are useful for treating a mood disorder. Optionally, the movement disorder is a depressive disorder, a bipolar disorders, or anxiety.

In one embodiment, the present methods and medicaments are useful for treating pain. Optionally, the pain is neurological pain or nociceptive pain. Optionally, the pain is associated with a movement disorder, a headache, a spasm disorder, arthritis, dystonia, peripheral pain, or muscle aching. For example, treatable pain can be associated with any disorder such as fibromyalgia or multiple sclerosis.

Pain that is treatable by the present medicament includes pain associated with any of the infections caused by herpes simplex virus type 1 and type 2 and herpes zoster.

Headaches that are treatable by the present medicament include any vascular headache, e.g., migraines, cluster headache, toxic headache, and headache caused by elevated blood pressure.

Headaches that are treatable by the present medicament also include tension headaches, postcoital headaches, exertional headaches, trigeminal neuralgia, atypical trigeminal neuralgia, type 2 trigeminal neuralgia, trigeminal autonomic cephalalgias, hortons neuralgia, and histamine headaches, and headaches secondary to head or neck trauma.

In one embodiment, the present methods and medicaments are used for appetite stimulation or to treat wasting and/or depressed appetite. Optionally the wasting and/or depressed appetite is associated with HIV, chemotherapy, anorexia, or Alzheimer's disease.

In one embodiment, the present methods and medicaments are useful for treating glaucoma.

In one embodiment, the present methods and medicaments are useful for treating nausea and/or vomiting. Optionally, the nausea and/or vomiting is associated with viral/microbial illness, HIV/AIDs, cancer, chemotherapy, radiation exposure, postoperative recovery, pregnancy, motion, or poisoning.

In one embodiment, the present methods and medicaments are useful for treating a subject with a disorder selected from: anorexia, alcohol use disorders, cancer, amyotrophic lateral sclerosis, glioblastoma multiforme, glioma, increased intraocular pressure, glaucoma, inflammatory bowel disorders, arthritis, dermatitis, Rheumatoid arthritis, systemic lupus erythematosus, inflammation, peripheral neuropathic pain, neuropathic pain associated with post-herpetic neuralgia, diabetic neuropathy, shingles, burns, actinic keratosis, oral cavity sores and ulcers, post-episiotomy pain, psoriasis, pruritis, contact dermatitis, eczema, bullous dermatitis herpetiformis, exfoliative dermatitis, mycosis fungoides, pemphigus, severe erythema multiforme (e.g., Stevens-Johnson syndrome), seborrheic dermatitis, ankylosing spondylitis, psoriatic arthritis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, post-traumatic osteoarthritis, synovitis, juvenile rheumatoid arthritis, major depressive disorder, depression, brain cancer, asthma, lung cancer, chronic obstructive pulmonary disease, opioid dependence, muscle tension, posttraumatic stress disorder, and bipolar disorder.

The citations provided herein are hereby incorporated by reference for the cited subject matter.

### EXAMPLES

### Example 1

### Treatment of Apnea with Marinol

The goal of the clinical trial was to evaluate oral dosing of THC in sleep apnea patients. One objective was to determine if low dosages of cannabinoids provide an effective treatment for apnea. Another objective was to evaluate the therapeutic window of oral cannabinoid in the treatment of sleep-related disorders such as apnea.

The trial comprised a single-center, randomized, double-blind, placebo-controlled dose escalation study of dronabinol in 22 patients with OSAS. The study began with a 7-day baseline/PAP-washout period, with polysomnography (PSG) performed on the final night. Subjects meeting inclusion/exclusion criteria were randomized to either placebo (N=5) or dronabinol (N=17) treatment.

The study drug (active or placebo) was taken 30 min before bed for 21 days. Overnight PSG was performed on treatment nights 7, 14 and 21. The initial nightly dose was 2.5 mg and was escalated, as tolerated, to 5 mg on day 8 and to 10 mg on day 15 of treatment. A blood sample was drawn immediately after each PSG for assay of the study drug and principal metabolites.

Sleep/activity/drug logs were maintained daily throughout the study. A Stanford Sleepiness Scale (SSS) was completed every 2 waking hours for the final two days of each 7-day baseline or treatment period.

The analysis of efficacy endpoints was performed using the Efficacy Evaluable population, defined as: all subjects completing a baseline PSG who received at least one dose of study medication, who did not miss more than 3 doses during, and who completed the PSG ending the first 7-day treatment period. Safety/tolerability analyses were performed using the All-Treated population, comprising all subjects who received at least one dose of study medication. All efficacy endpoints were assessed as the change from baseline measurement of the same parameter. For example, efficacy for AHI was examined by subtracting (for each subject) the AHI measured during PSG at the end of the baseline period from the AHE measured at the end of the relevant treatment period (in both active and placebo groups). Thus a decrease in AHI with treatment is represented by a negative value for ΔAHI (change from baseline).

Results for Arousal Index (arousals per hour of sleep) are shown in Table 5. Surprisingly, these data demonstrate that oral cannabinoids provide a therapeutic benefit to sleep continuity in apnea patients, even in reduced amounts. For example, these data support treatment by administering oral doses of less than 70 mg, 60 mg, or even less than 50 mg, such as 0.1, 0.5, 1.0, or 2.5 mg-20 mg doses.

Surprisingly, these data also demonstrate that sleep apnea may be treated with orally administered cannabinoids without causing (or without substantially causing) side effects associated with certain cannabinoids and/or without causing (or without substantially causing) side effects once the subject has awoken (e.g. post treatment window).

These results, and those of the subsequent Examples, also support the applicants' invention of methods of treating sleep apnea with a reduced dose (e.g. about 0.1-about 20 mg) of an immediate release cannabinoid.

TABLE 5
Arousal Index (Arousals/Hour of Sleep)

| Parameter | 2.5 mg | 10 mg |
|---|---|---|
| Number of Subjects | 17 | 8 |
| Number of Observations | 23 | 8 |
| Mean Change with treatment | −4.8 | −2.5 |
| vs Placebo | | |
| Significance vs Placebo | 0.49 | 0.79 |

### Example 2

### Marinol for Apnea

### Comparing Early and Late Treatment Windows

The study from Example 1 was further analyzed with respect to Arousal Index during the early treatment window (i.e., T₀-T₄) and the late treatment window (i.e., T₅-T₈).

TABLE 6
Arousal Index, Early and Late Treatment Window

| | 2.5 mg | 10 mg |
|---|---|---|
| 1^{st} Half of Night | | |
| Number of Subjects | 17 | 8 |
| Number of Observations | 23 | 8 |
| Mean Change with | −20.0 | −21.9 |
| treatment vs Placebo | | |
| Significance vs Placebo | 0.067 | 0.11 |
| 2^{nd} Half of Night | | |
| Number of Subjects | 17 | 8 |
| Number of Observations | 23 | 8 |
| Mean Change with | 0.6 | 5.0 |
| Treatment vs Placebo | | |
| Significance vs Placebo | 0.93 | 0.56 |

### Example 3

### Marinol for Apnea

### 75% Reduction Analysis

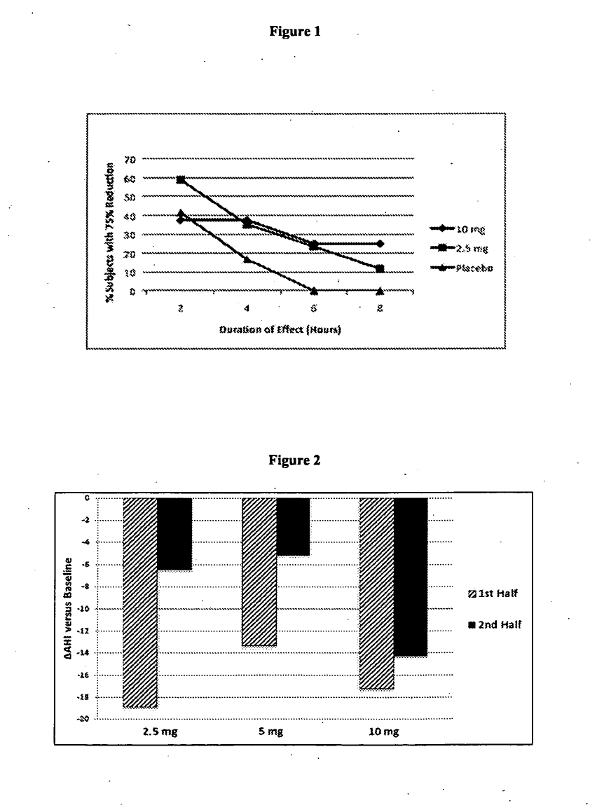

The study from Example 1 was further analyzed with respect to the percentage of subjects demonstrating a 75% reduction in the AHI for 2-, 4-, 6-, and 8-hour consecutive intervals. As shown in FIG. 1, a dose of 2.5 mg (line with square data points) resulted in greater than 60% of the subjects showing a ≧75% reduction (versus baseline) in AHI for at least 2 consecutive hours. In contrast, a dose of 10 mg (line with diamond data points) resulted in fewer than 30% of the subjects showing a 2-hour reduction in AHI of ≧75%. This same phenomenon was seen with respect to a four-hour response interval. Thus, for a 2 and 4 hour treatment window, 2.5 mg of Marinol was more effective in these patients than a 10 mg dose. In contrast to the expected sigmoidal dose-response curve that typifies most drug therapies, THC effect demonstrated here is consistent with a non-monotonic response of the inverted U. Thus, a superior medicament of the present invention produces a threshold plasma THC concentration but does not reach the decreasing response portion of the dose curve.

### Example 4

### Marinol for Apnea

Dose and Time Dependence

The study from Example 1 was further analyzed for efficacy and dose response of THC with respect to AHI during early (T₀-T₄) and late (T₅-T₈) treatment windows. In the results are shown in FIG. 2; the early treatment window is indicated by stippled bars and the late treatment window is indicated by solid bars.

The orally administered instant release cannabinoid provided remarkable efficacy during the early treatment window. Consistent with Example 3, these results further unexpectedly show that 2.5 mg of Marinol was superior to 10 mg which was superior to 5 mg. In contrast, in the late treatment window, 10 mg of Marinol was superior to 2.5 mg and 5 mg.

### Example 5

### Marinol for Apnea

Establishing the Therapeutic Window

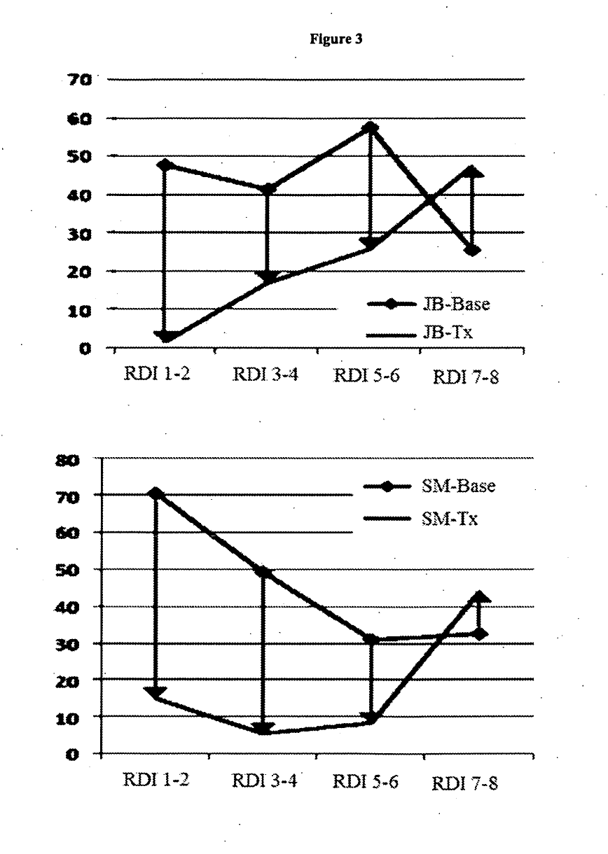

The apnea-hypopnea index (AHI) during a treatment window was calculated for two examplary patients (“JB” and “SM”) for hours T₁-T₂ (RDI1-2), T₃-T₄ (RDI1-2), T₅-T₆ (RDI1-2), and T₇-T₈ (RDI1-2). The patients had each taken a single 2.5 mg dose of Marinol 30 minutes before bed. The results, as shown in FIG. 3, are of the baseline (lines with diamond data points) and the treatment (lines without symbols). A single 2.5 mg immediate release dose of cannabinoid (Marinol) provided a significant therapeutic effect during an early treatment window. These data are consistent with the arousal index data presented in Table 6.

### Example 6

### Increasing Efficacy with Weeks of Treatment

The study from Example 1 was further analyzed for efficacy with prolonged exposure (i.e. weeks of once per day treatment) with 2.5 mg of Marinol. The results are shown in FIG. 6, where the first bar in each pair is the first treatment window (T₀ₕᵣ to T₄ₕᵣ) and the second bar of each pair of bars is the late treatment window (T₄ₕᵣ to T₆ₕᵣ). Initially (i.e. during the first one week period of treatment), subjects demonstrated remarkable decrease in AHI during an early treatment window but substantially diminished efficacy during the late treatment window.

By the third week of treatment, two remarkable results were observed. First, the substantial efficacy observed during the early treatment window continued or increased. Second, the efficacy during the late treatment window (which was diminished during the first week of treatment) was of a similar magnitude as during the early treatment window.

These data demonstrate the remarkable efficacy of reduced dose medicaments of the present invention, efficacy during an extended treatment window (e.g. 8 hours), and increasing efficacy with prolonged exposure.

### Example 7

### Oral Dosing

For the purpose of comparing oral medicaments (i.e., providing a comparator), a medicament is orally administered to one or more subjects. The various factors affecting ADME (absorption, distribution, metabolism, and excretion) of the drug are standardized. For example, the patient is optionally a fasted patient and optionally falls asleep within one of 15 minutes or 30 minutes of laying down for bed. The same subjects are used for comparing different medicaments (after providing an appropriate wash-out period).

Plasma levels of the drug (e.g. THC) and metabolites thereof (e.g. 11-OH-THC) are taken at regular intervals (e.g. every 30 minutes) during a treatment window (e.g. from T₀ to T₈ₕᵣ.

Therapeutic responses are recorded throughout the entire treatment window.

Therapeutic responses are correlated with pharmacokinetic parameters of the drug or metabolites thereof. The pharmacokinetic parameters include one or more of: plasma concentration and AUC (at various times).

### Example 8

### Predictable Plasma Levels

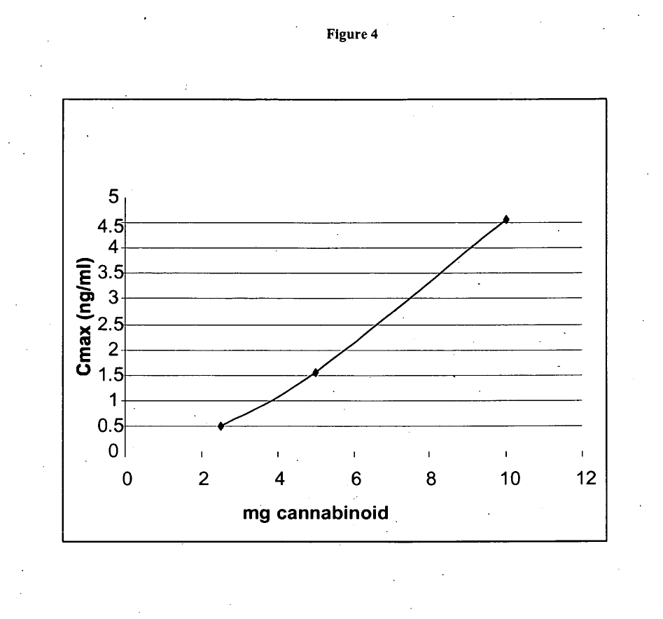

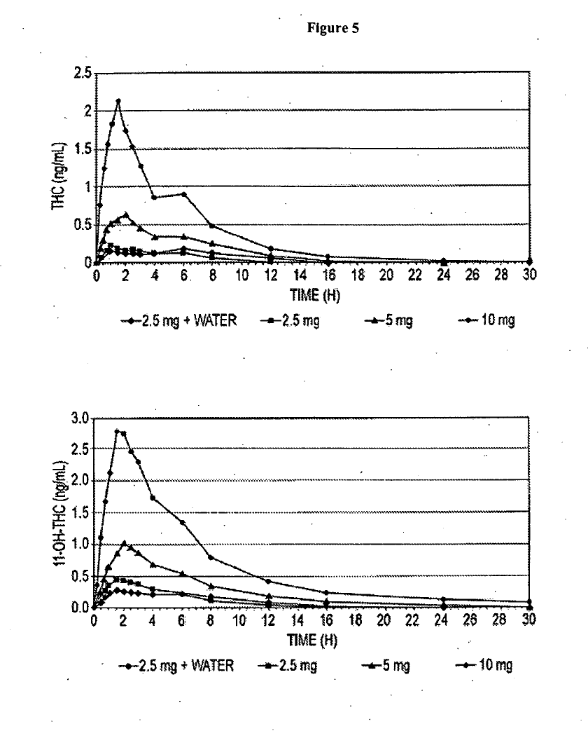

FIG. 4 depicts the relationship of Cmax (ng/ml) and cannabinoid amount (mg) for an immediate release dosage compartment (Marinol formulation). As can be seen from FIG. 4, given the drug dose, plasma levels such as Cmax are predictable from an immediate release dosage compartment.

### Example 9

### Medicament

### Plurality of Pellets, Continuous Release

A medicament is provided comprising an immediate release medicament of the present invention. The medicament in this example is a plurality of solid pellets (or microspheres).

TABLE 7
Pellet in Capsule
10 mg Dronabinol (or other THC) Capsule

| | | | |
|---|---|---|---|
| No. | Ingredient | mg/capsule | % w/w |

Immediate Release Pellets/Compartment

| | | | |
|---|---|---|---|
| 1 | Dronabinol | 2.5 | 1.16 |
| 2 | Sodium Lauryl Sulfate | 2.5 | 1.16 |
| 3 | Neusilin US2 (Magnesium | 20.0 | 9.30 |
| | Aluminometasilicate) | | |
| 4 | Avicel PH101 (Microcrystalline | 25.0 | 11.63 |
| | Cellulose) | | |
| | SubTotal | 50.0 | 23.3 |

Manufacturing Process for Dronabinol Pellets.
A) Dissolve Dronabinol (1+5) in Ethanol (200 proof) in a suitable tank and mixer.
B) Disperse Sodium Lauryl Sulfate (2+6) into (A). Continue to mix.
C) Charge Neusilin (3+7) to a high shear granulator.
D) While mixing the Neusilin, add the dispersion from (B). Mix until (B) is suitably dispersed.
E) Transfer the wet contents of the high shear granulator to a tray dryer.
F) Dry at 50 C (+/−10 C) to remove the Ethanol.
G) Collect the dried material from the trays and pass through a No. 20 mesh screen to deagglomerate.
H) Charge the Dronabinol-Loaded Neusilin (G) to a high shear granulator.
I) Charge the required quantity of Avicel PH101 (4+8) to the high shear granulator.
J) Mix the contents in the high shear granulator for 5 minutes.
K) While mixing, add sufficient purified water to wet mass.
L) Transfer the wet mass (K) to a suitable cold mass extruder fitted with a dome screen of suitable size (e.g. 0.8 mm-1.2 mm).
M) Extrude the wet mass to collect strands.
N) Load the collected strands (M) into a marumerizer. Operate the marumerizer to produce pellets.
O) Extrude/marumerize the entire wet mass (K) from the high shear granulator.
P) Transfer the wet pellets (O) to a fluid bed or tray dryer and dry.
Q) Collect the dried pellets (P) from the dryer into a suitable container.
R) Using screens, collect pellets that pass through a No. 18 mesh screen but are retained on a No. 30 mesh screen.

Encapsulation
Z) Using a suitable encapsulator, fill suitably sized two piece hard capsule shells with 50 mg of Dronabinol IR 10% w/w Pellets (18-30 mesh) and 165 mg of Dronabinol DR Pellets.

### Example 10

### Matrix Tablet

A medicament is provided comprising an immediate release medicament of the present invention. The immediate release medicament is a distinct solid matrix layer without release modifying excipients. The medicament provided in this Example is a solid, adsorbate to facilitate solids handling of dronabinol.

TABLE 8
Matrix Tablet
Dronabinol (or other THC) Tablet

| | | | |
|---|---|---|---|
| No. | Ingredient | mg/tablet | % w/w |

Immediate Release Layer/Compartment

| | | | |
|---|---|---|---|
| 1 | Dronabinol | 2.5 | 0.61 |
| 2 | Sodium Lauryl Sulfate | 2.5 | 0.61 |
| 3 | Fujicalin (Dicalcium Phosphate Anhydrous) | 50.0 | 12.14 |
| 4 | Prosolv SMCC 90HD (Silicified | 39.5 | 9.59 |
| | Microcrystalline Cellulose) | | |
| 5 | Croscarmellose Sodium | 5.0 | 1.21 |
| 6 | Magnesium Stearate | 0.5 | 0.12 |
| | SubTotal | 100.0 | 24.27 |

Outer Trade Dress Coating (Non release modifying)

| | | | |
|---|---|---|---|
| 14 | Opadry Colored Coating System - PVA | 12.0 | 2.91 |
| | Based | | |
| | Total Tablet Weight | 412.0 | 100 |

Manufacturing Process for Dronabinol Loading onto Fujicalin (Adsorbate Formation)
A) Dissolve Dronabinol (1+7) in Ethanol (200 proof) in a suitable tank with a mixer.
B) Disperse Sodium Lauryl Sulfate (2+8) into (A). Continue to mix.
C) Charge Fujicalin (3+9) to a high shear granulator.
D) While mixing the Fujicalin, add the dispersion from (B). Mix until (B) is suitably dispersed.
E) Transfer the wet contents of the high shear granulator to a tray dryer.
F) Dry at 50 C (+/−10 C) to remove the Ethanol.
G) Collect the dried material from the trays and pass through a No. 20 mesh screen to deagglomerate.
H) Store in an HPDE drum double lined with PE bags and desiccants as required.

Immediate Release Layer Blend/Compartment. The prepared powder blend provides immediate release of the dronabinol from the compressed tablet.
I) Charge the required quantity of the Dronabinol-Loaded Fujicalin (H) into a tumble blender after passing through a No. 20 mesh screen.
J) Charge the required quantity of Prosolv (4) to the tumble blender after passing through a No. 20 mesh screen.
K) Close the blender and blend for 15 minutes.
L) Open the blender and charge the required quantity of Croscarmellose Sodium (5) after passing through a No. 20 mesh screen.
M) Close the blender and blend for 5 minutes.
N) Open the blender and charge the required quantity of Magnesium Stearate (6) after passing through a No. 20 mesh screen.
O) Close the blender and blend for 2 minutes.
P) Collect the IR Layer Blend (O) from the blender into an HDPE drum double lined with PE bags and desiccants as required.

Tablet Compression.
Q) Using a dual/bi-layer tablet press, compress tablets containing 100 mg of the IR Layer Blend/Compartment.
R) Collect the Dronabinol Tablets into HDPE drums lined with PE bags and desiccants as required.

Tablet Coating (Non-release modifying trade dress)
AA) Prepare the Opadry Coating System as an 18% w/w dispersion in water using a suitable tank and mixer.
AB) Load the Dronabinol Tablets into a suitable tablet coater.
AC) Apply a 3% weight gain of the Opadry Coating dispersion to the tablets.
AD) Collect the coated tablets from the tablet coater into HDPE drums lined with PE bags and desiccants as required.

We claim:

1. A method of treating a subject with a cannabinoid-sensitive disorder comprising treating the subject for at least 30 days with a plurality of administrations of an oral medicament, wherein:
the oral medicament comprises a cannabinoid in an amount of about 0.05 mg to about 2.0 mg; and
the subject is a human.

2. The method of claim 1 wherein the cannabinoid-sensitive disorder is a sleep apnea.

3. The method of claim 1 wherein the medicament is a liquid, optionally wherein the liquid is a lipophilic medium or oil.

4. The method of claim 3 wherein the liquid is a lipophilic medium or oil, and wherein the lipophilic medium or oil is cottonseed oil, sesame oil, coconut oil or peanut oil.

5. The method of claim 1 wherein the medicament is an instant release medicament.

6. The method of claim 1 wherein the medicament is in the form of a tablet, a capsule, a sachet, sprinkles, or a suspension.

7. The method of claim 1 wherein:
the cannabinoid-sensitive disorder is a sleep apnea;
the plurality of administrations are carried out daily about 30 to about 60 minutes before a sleep cycle;
the method further comprises a subsequent step of administering the oral medicament to the subject about 30 to about 60 minutes before a sleep cycle.

8. The method of claim 7 wherein the subject has a reduction in the number of apnea events when compared to the average number of apnea events experienced before administering the method of claim 7.

9. The method of claim 7 wherein the subject has a reduction in the AHI index when compared to the average AHI index experienced before administering the method of claim 7.

10. The method of claim 7 wherein the subject has a reduction in the arousal index when compared to the average arousal index experienced before administering the method of claim 7.

11. The method of any one of claims 8-10 wherein the reduction occurs at least in the period of about 4 to about 6 hours after administration.

12. The method of any one of claims 1-10 wherein:
the medicament further comprises at least one agent selected from the group consisting of an anti-apnea agent, an anti-convulsant, an analgesic, an anti-anxiety or anxiolytic agent, and an appetite stimulant; and
the at least one agent is not a cannabinoid.

13. The method of claim 7 wherein the medicament further comprises an anti-apnea agent other than a cannabinoid.

14. The method of any one of claims 1-6 wherein the cannabinoid-sensitive disorder is selected from the group consisting of sleep apnea, anxiety, stress, a mood disorder, pain, neuropathic pain, nociceptive pain, headache, nausea, glaucoma, a seizure disorder, anorexia-cachexia syndrome, and combinations thereof.

15. The method of any one of claims 1-6 wherein the cannabinoid-sensitive disorder is anxiety, stress, or a mood disorder, and the medicament further comprises an anxiolytic other than a cannabinoid.

16. The method of any one of claims 1-6 wherein the cannabinoid-sensitive disorder is neuropathic pain, nociceptive pain, or headache, and the medicament further comprises an analgesic other than a cannabinoid.

17. The method of any one of claims 1-6 wherein the cannabinoid-sensitive disorder is nausea, and the medicament further comprises an anti-emetic other than a cannabinoid.

18. The method of any one of claims 1-6, wherein the cannabinoid-sensitive disorder is glaucoma; and the medicament further comprises an anti-glaucoma therapeutic agent other than a cannabinoid.

19. The method of any one of claims 1-6, wherein the cannabinoid-sensitive disorder is a seizure disorder and the medicament further comprises an anti-convulsant other than a cannabinoid.

20. The method of any one of claims 1-6, wherein the cannabinoid-sensitive disorder is anorexia-cachexia syndrome and the medicament further comprises an appetite stimulant.

21. The method of any one of claims 1-10, wherein the cannabinoid is dronabinol.

22. A method of establishing an optimal dose of a medicament for a subject with a cannabinoid-sensitive disorder comprising the steps of:
(a) administering a first dose of the medicament for a first treatment period, wherein the first dose comprises the medicament in an amount of about 0.05 mg to about 2.0 mg;
(b) administering a second dose of the medicament for a second treatment period wherein the second dose is increased compared to the first dose;
(c) administering a third dose of the medicament for a third treatment period wherein the third dose is increased compared to the second dose;
(d) optionally administering a fourth dose of the medicament for a fourth treatment period wherein the fourth dose is increased compared to the third dose;
(e) if step d is performed, optionally administering a fifth dose of the medicament for a fifth treatment period wherein the fifth dose is increased compared to the fourth dose;
(f) if step e is performed, optionally administering a sixth dose of the medicament for a sixth treatment period wherein the sixth dose is increased compared to the fifth dose; and
(g) if step f is performed, optionally administering a seventh dose of the medicament for a seventh treatment period wherein the seventh dose is increased compared to the sixth dose,
wherein the medicament is an oral cannabinoid medicament.

23. The method of claim 22, wherein a clinically-relevant metric is obtained at one or more of steps (a)-(g) and a treatment-related side effect is assessed at one or more of steps (a)-(g).

24. The method of claim 23 further comprising a step (h) of comparing said clinically-relevant metric and said treatment-related side effect and the step of selecting a dose for chronic treatment.

25. The method of any one of claims 22-24 wherein the treatment period is about 1 day to about 30 days.

26. The method of any one of claims 22-24 wherein the dose increase of each of the doses is about 10% to about 200%.

27. The method of any one of claims 22-24 wherein the dose increase of each of the doses is about 25% to about 200%.

28. The method of any one of claims 22-24 wherein the first dose is about 0.1 to about 1 mg.

29. The method of claim 23 further comprising a step (i) of administering a decreased dose of the medicament for a treatment period and comparing a clinically-relevant metric and treatment-related side effects during this reduced dose step and the previous dose step and optionally further comprising the step of selecting a dose for chronic treatment.

30. The method of any one of claims 22-24 and 29 further comprising a step (i) of administering a decreased dose of the medicament for a treatment period and comparing a clinically-relevant metric and treatment-related side effects during this reduced dose step and the previous dose step and optionally further comprising the step of selecting a dose for chronic treatment.

31. The method of claim 29 further comprising the step (j) which comprises the step of repeating step (i) and optionally further comprising the step of selecting a dose for chronic treatment.

32. The method of claim 30 further comprising the step (j) which comprises the step of repeating step (i) and optionally further comprising the step of selecting a dose for chronic treatment.

33. The method of any one of claims 22-24 and 29 wherein the cannabinoid-sensitive disorder is sleep apnea.

34. A kit for performing the method of any one of claims 22-24 and 29 comprising a sufficient number of medicament units for said method, and optionally comprising instructions for performing said method.

35. A method of treating a subject with a cannabinoid-sensitive disorder comprising:
a. treating the subject during a first treatment period with a plurality of administrations of a first dose of an oral medicament; and
b. treating the subject during a second treatment period with a plurality of administrations of a second dose of an oral medicament;
wherein:
the subject is a human;
the oral medicament comprises a cannabinoid;
the second treatment period is subsequent to the first treatment period;
the second dose is less than the first dose; and
therapeutic efficacy during the second treatment period is not reduced compared to the therapeutic efficacy during the first treatment period.

36. The method of claim 35, wherein the cannabinoid-sensitive disorder is selected from the group consisting of a sleep apnea, anxiety, stress, a mood disorder, pain, neuropathic pain, nociceptive pain, headache, nausea, glaucoma, a seizure disorder, anorexia-cachexia syndrome, and combinations thereof.

37. The method of claim 36, wherein the first treatment period is at least about one week, at least about two weeks, at least about 1 month, or at least about 1 year.

38. The method of claim 35, wherein the first treatment period is at least about one week, at least about two weeks, at least about 1 month, or at least about 1 year.

39. The method of claim 35, wherein the cannabinoid is dronabinol.

40. The method of claim 36, wherein the cannabinoid is dronabinol.

41. The method of claim 37, wherein the cannabinoid is dronabinol.

42. The method of claim 38, wherein the cannabinoid is dronabinol.

43. The method of any of claims 35-42, wherein each of the first dose and the second dose comprises less than about 25 mg of the cannabinoid.

44. The method of claim 43, wherein each of the first dose and the second dose comprises less than about 10 mg of the cannabinoid.

45. The method of claim 43, wherein each of the first dose and the second dose comprises less than about 5 mg of the cannabinoid.

46. The method of claim 43, wherein each of the first dose and the second dose comprises less than about 2.5 mg of the cannabinoid.

47. The method of claim 43, wherein each of the first dose and the second dose comprises about 0.05 mg to about 2.0 mg of the cannabinoid.

48. The method of claim 44, wherein each of the first dose and the second dose comprises about 0.05 mg to about 5.0 mg of the cannabinoid.

49. A method of treating a subject with a sleep apnea disorder comprising:
a. providing a plurality of equal doses, each comprising about 0.05 mg to about 25 mg of a cannabinoid;
b. administering one of said plurality of doses to said subject about 30 to about 60 minutes before a sleep cycle;
c. repeating step b. daily for a first treatment period, wherein in the first treatment period, the subject experiences apneic events during a window of the sleep cycle; and
d. repeating step b. daily at least until the subject exhibits a reduction, compared to the first treatment period, in the number of apneic events during the window of the sleep cycle, optionally wherein the reduction in the number of apneic events is a reduction by 50% or 100%.

50. The method of claim 49, wherein the window is T₀ₕᵣ to T₈ₕ, T₀ₕᵣ to T₆ₕ, or T₄ₕᵣ to T₆ₕᵣ.

51. The method of claim 49 or 50, wherein the first treatment period is at least about one week, at least about one month, at least about two months, or at least about three months.

52. The method of claim 51, wherein the cannabinoid is dronabinol.

53. The method of claim 49 or 50, wherein the cannabinoid is dronabinol.

54. The method of claim 49, wherein each of the plurality of doses comprises about 0.05 to about 5 mg, about 0.05 to about 2.5 mg, or about 0.05 to about 2.0 mg of the cannabinoid.

55. The method of claim 50, wherein each of the plurality of doses comprises about 0.05 to about 5 mg, about 0.05 to about 2.5 mg, or about 0.05 to about 2.0 mg of the cannabinoid.

56. The method of claim 51, wherein each of the plurality of doses comprises about 0.05 to about 5 mg, about 0.05 to about 2.5 mg, or about 0.05 to about 2.0 mg of the cannabinoid.

57. The method of claim 52, wherein each of the plurality of doses comprises about 0.05 to about 5 mg, about 0.05 to about 2.5 mg, or about 0.05 to about 2.0 mg of the cannabinoid.

58. The method of claim 53, wherein each of the plurality of doses comprises about 0.05 to about 5 mg, about 0.05 to about 2.5 mg, or about 0.05 to about 2.0 mg of the cannabinoid.

59. A method of treating a subject with a sleep apnea disorder comprising:
a. administering to the subject for a first treatment period, a fixed daily dose of an oral medicament comprising about 0.05 mg to about 25 mg of a cannabinoid;
b. administering the fixed daily dose to the subject for a second treatment period,
wherein:
the first treatment window is at least about 14 days;
the second treatment window is at least 7 days; and
the subject experiences greater average daily therapeutic efficacy during the second treatment window compared to the first treatment window.

60. The method of claim 59, wherein the first treatment period is 20 days, optionally wherein the second treatment period is 10 days.

61. The method of claim 59, wherein the first treatment period at least 50 days, optionally wherein the second treatment period is 10 days.

62. The method of claim 61, wherein the first treatment period is 50 days.

63. The method of claim 59, wherein the first treatment period is at least 80 days, optionally wherein the second treatment period is 10 days.

64. The method of claim 63, wherein the first treatment period is 80 days.

65. The method of claim 59, wherein the first treatment period is 30 days and the second treatment period is 60 days.

66. The method of any of claims 59-65, wherein the medicament comprises about 0.05 mg to about 5 mg of a cannabinoid.

67. The method of any of claims 59-65, wherein the medicament comprises about 0.05 mg to about 2.5 mg of a cannabinoid.

68. The method of any of claims 59-65, wherein the medicament comprises about 0.05 mg to about 2.0 mg of a cannabinoid.

69. The method of claim 66, wherein the cannabinoid is dronabinol.

70. The method of claim 67, wherein the cannabinoid is dronabinol.

71. The method of claim 68, wherein the cannabinoid is dronabinol.

72. The method of any of claims 59-65, wherein the cannabinoid is dronabinol.
